(19) United States
(12) Patent Application Publication
(10) Pub. No.: US 2003/0109443 A1
(43) Pub. Date: Jun. 12, 2003

(54) Diagnosing, treating, and preventing cancer using cables

(76) Inventors: Bo R. Rueda, Windham, NH (US); Lawrence R. Zukerberg, Newton, MA (US); Chin-Lee Wu, Newton, MA (US)

Correspondence Address: CLARK & ELBING LLP, 101 FEDERAL STREET, BOSTON, MA, 02110, US

(21) Appl. No.: 10/262,480

(22) Filed: Oct. 1, 2002

Related U.S. Application Data
(60) Provisional application No. 60/326,465, filed on Oct. 1, 2001.
(60) Provisional application No. 60/356,685, filed on Feb. 14, 2002.

(51) Int. Cl. C12Q 1/68; G01N 33/574; A61K 38/18; A61K 48/00

(52) U.S. Cl. 514/12; 514/44; 435/6; 435/7.23

(57) ABSTRACT

Disclosed herein is are methods of diagnosing, methods for determining the prognosis for treatment of, and methods for identifying candidate compounds for treating, stabilizing, or preventing cancer, for example, endometrial cancer. These methods utilize the novel tumor suppressor protein, Cables.

### CROSS REFERENCE TO RELATED APPLICATIONS

This application claims the benefit of the filing dates of provisional application U.S. Ser. No. 60/326,465, filed Oct. 1, 2001, and U.S. Ser. No. 60/356,685, filed Feb. 14, 2002.

### FIELD OF THE INVENTION

The field of the invention is cancer diagnosis, treatment, and therapeutics screening. In particular, the invention relates to methods for identifying candidate compounds that modulate the gene expression or polypeptide activity of a novel tumor suppressor protein, termed Cables. The invention further relates to methods for diagnosing and treating Cables-related cancers.

### BACKGROUND OF THE INVENTION

Cyclin dependent kinases (cdks) comprise a family of serine/threonine protein kinases that have been shown to be key regulators of cell cycle progression. The cdks require association with regulatory subunits known as cyclins for activation. In addition to binding cyclins, post-translational phosphorylation and dephosphorylation events regulate cdk activity. Phosphorylation of the threonine residue in the T loop (T160 on cdk2 or T161 on cdc2) by cdk-activating kinase (CAK) is an obligatory step in kinase activation, and a threonine to alanine mutation of this residue renders the cdk inactive. On the other hand, phosphorylation of the threonine 14 and tyrosine 15 (Y15) residues by the Wee1 family of dual specificity kinases is inhibitory for the cdks, and dephosphorylation of these residues by the Cdc25 family of phosphatases coincides with cdk activation.

There is a clear connection between cdk regulation and cancer. Overexpression of certain cyclins contribute to cell transformation. A mutant cyclin A protein, that lacked the cyclin destruction box as a result of hepatitis B virus insertion, was implicated in the tumorigenesis of a hepatoma. Cyclin D1 has been identified as the Bcl-1 proto-oncogene that is overexpressed by translocation or amplification in parathyroid adenomas, lymphomas, squamous cell carcinomas of the head and neck, and breast tumors. Some of the cdk inhibitory molecules are tumor suppressor genes, such as p16, which has alternatively been termed Multiple Tumor Suppressor 1 (MTS1). Another cdk interactor, p21, is activated by p53 and blocks cdk activity and cell cycle progression. In cells lacking functional p53, the failure to induce p21 after DNA damage may contribute to the increased incidence of chromosomal abnormalities and genetic instability in transformed cells. Similarly, many tumor cell lines proceed through the cell cycle with damaged DNA suggesting there is a defect in the regulation of cdk2 T14/Y15 phosphorylation. The defect could lie in the Wee1 mediated cdk2 T14/Y15 phosphorylation or Cdc25 phosphatase, which removes the cdk inhibitory phosphorylations. Cdc25 has been implicated as an oncogenic protein in colorectal, stomach, breast, and lung cancers.

### SUMMARY OF THE INVENTION

In general, in a first aspect, the invention features a method of diagnosing a cancer-related condition either by measuring Cables expression or Cables activity in a sample from a subject, wherein a decrease in Cables expression or Cables activity in the sample, relative to the amount of Cables expression or Cables activity in a control sample, indicates that the subject has a cancer-related condition or a propensity to develop a cancer-related condition.

In one embodiment, the sample also contains either a cdk2 polypeptide, a Wee1 polypeptide, or both. In another embodiment, Cables activity is determined by measuring Wee1-mediated tyrosine phosphorylation of cdk2. In yet another embodiment, Cables expression is determined by measuring the amount of Cables mRNA or polypeptide. In another embodiment, Cables expression or Cables activity in the sample is compared to Cables expression or Cables activity in control samples from both subjects having a cancer-related condition and subjects not having a cancer-related condition.

In a related aspect, the invention features another method for diagnosing a cancer-related condition that involves detecting the absence of a Cables gene or a mutation in a Cables gene, wherein the absence of a Cables gene or a mutation in a Cables gene indicates a cancer-related condition or a propensity thereto in the subject.

In one embodiment of this method, the mutation of the Cables gene results in expression of a truncated Cables polypeptide. In another embodiment, the human Cables gene is located on chromosome 18, region q11.2-q12.1.

In yet another related aspect, the invention features a third method for diagnosing a cancer-related condition that involves detecting the presence of hypermethylation of CpG islands in a Cables promoter of a subject, wherein the presence of hypermethylation indicates a cancer-related condition or a propensity thereto in the subject.

The invention also features a method of identifying a subject at increased risk of developing a cancer-related condition by determining whether there is an alteration in a Cables nucleic acid molecule. The presence of an alteration indicates that the subject is at increased risk of developing a cancer-related condition.

In a preferred embodiment, the diagnosis method further involves assessing the effect of progestin on a tissue sample of a subject at increased risk of developing a cancer-related condition. An increase in Cables expression or a decrease in cellular proliferation in the cells of the tissue sample exposed to progestin indicates a favorable diagnosis. An unfavorable diagnosis is made if exposure of the tissue sample to progestin results in little or no increase in Cables expression or if no decrease in cellular proliferation is observed.

In a second general aspect, the invention features methods for determining the prognosis for treatment of a cancer-related condition in a subject. The first such method involves measuring the level of Cables expression or Cables activity in a sample from the subject, wherein an increase or decrease in the Cables expression or Cables activity in the sample, relative to the amount of Cables expression or Cables activity in a control sample, determines the prognosis for treatment of a cancer-related condition in the subject.

In a preferred embodiment, the level of Cables expression or Cables activity in the sample is compared to the level of Cables expression or Cables activity in control samples from both subjects having the cancer-related condition and subjects not having the cancer-related condition. Typically, a decrease in the level of Cables expression or Cables activity indicates a negative prognosis for the treatment of the cancer-related condition.

The invention further features a second method for determining the prognosis for treatment of a cancer-related condition in a subject by detecting the absence of a Cables gene or a mutation in a Cables gene in the subject. The absence of the Cables gene or a mutation in the Cables gene indicates a negative prognosis. In one embodiment of this method, a mutation in the Cables gene results in expression of a truncated Cables polypeptide. In another embodiment, the human Cables gene is located on chromosome 18, region q11.2-q12.1.

In a related aspect, the invention features yet another method for the prognosis for treatment of a cancer-related condition in a subject. This method involves detecting the presence of hypermethylation of CpG islands in a Cables promoter. The presence of hypermethylation of CpG islands in a Cables promoter indicates a negative prognosis.

In a preferred embodiment, the prognosis for treatment of a cancer-related condition in a subject can also be determined by assessing the effect of progestin on a tissue sample of a subject having a cancer-related condition. An increase in Cables expression or a decrease in cellular proliferation in the cells of the tissue sample exposed to progestin indicates a favorable prognosis. An unfavorable prognosis is made if exposure of the tissue sample to progestin results in little or no increase in Cables expression or if no decrease in cellular proliferation is observed.

In a third general aspect, the invention further features methods for identifying candidate compounds for treating, stabilizing, or preventing cancer-related conditions. The first screening method involves contacting a cell or in vitro sample having a Cables nucleic acid molecule (e.g., Cables cDNA or Cables mRNA) or polypeptide with a candidate compound and measuring a biological parameter of said Cables nucleic acid molecule or polypeptide. A candidate compound that increases a biological parameter of a Cables nucleic acid molecule or polypeptide, relative to the biological parameter of a Cables nucleic acid molecule or polypeptide in a cell or in vitro sample not contacted with the candidate compound, is a candidate compound for treating, stabilizing, or preventing a cancer-related condition.

In a preferred embodiment, the cell or in vitro sample contains a Cables nucleic acid molecule and the biological parameter that is measured is the level of transcription or translation of the Cables nucleic acid molecule. In a second preferred embodiment, the cell or in vitro sample contains a Cables polypeptide and the biological parameter that is measured is the level of biological activity of the Cables polypeptide.

In yet another preferred embodiment, measuring the biological parameter further includes measuring the amount of Cables mRNA or polypeptide in the cell or in vitro sample, in which an increase in the amount of the Cables mRNA or polypeptide, relative to the amount of Cables mRNA or polypeptide in a control cell or in vitro sample not contacted with the compound, indicates a candidate compound for treating, stabilizing, or preventing said cancer-related condition.

In another preferred embodiment, the cell or in vitro sample also contains either a cdk2 polypeptide, a Wee1 polypeptide, or both. In this embodiment, the biological activity of a Cables polypeptide is determined by measuring Wee1-mediated tyrosine phosphorylation of cdk2, and an increase in phosphorylation of cdk2 (for example, at tyrosine 15) indicates a candidate compound for treating, stabilizing, or preventing a cancer-related condition. In other preferred embodiments, the cell is a transgenic cell expressing a heterologous Cables polypeptide, for example, a transgenic cell in a trangenic animal.

In a related aspect, the invention features another method for identifying a candidate compound for treating, stabilizing, or preventing a cancer-related condition. This method involves contacting a cell or in vitro sample having a Cables gene or a Cables reporter construct and measuring the level of expression of the Cables gene or Cables reporter construct. A candidate compound that increases expression of the Cables gene or Cables reporter construct, relative to expression of the Cables gene or Cables reporter construct in a control cell or in vitro sample not contacted with the candidate compound, is a candidate compound for treating stabilizing, or preventing a cancer-related condition.

In one preferred embodiment, expression of the Cables gene or Cables reporter construct is measured by assaying the level of mRNA transcribed from the Cables gene or Cables reporter construct. In another preferred embodiment, expression of the Cables gene or Cables reporter construct is measured by assaying the level of polypeptide translated from the Cables gene or Cables reporter construct.

In preferred embodiments of each of the above screening methods, the Cables gene or Cables polypeptide is or is derived from a human Cables gene or human Cables polypeptide.

In a fourth aspect, the invention features a method of treating, stabilizing, or preventing a cancer-related condition in a subject by administering a Cables polypeptide, or a fragment thereof, a compound that increases Cables polypeptide expression or biological activity, or a Cables polypeptide-expressing nucleic acid molecule to the subject in an amount sufficient to treat, stabilize, or prevent the cancer-related condition. In a preferred embodiment, progestin is administered in combination with the Cables polypeptide, or fragment thereof, a compound that increases Cables polypeptide expression or biological activity, or a Cables polypeptide-expressing nucleic acid molecule. In another preferred embodiment, the Cables-polypeptide expressing nucleic acid molecule includes the human Cables nucleic acid provided in SEQ ID NO: 1. In another preferred embodiment, the Cables nucleic acid molecule is in an adenoviral vector that is administered to the subject.

In various preferred embodiments of any of the above methods for diagnosis, risk assessment, prognosis, compound screening, or therapy, the subject is a mammal, for example, a human; and the cancer-related condition is prostate cancer, ovarian cancer, colorectal cancer (e.g., colorectal adenocarcinoma), stomach cancer, lung cancer, esophageal cancer, head cancer, neck cancer, bladder cancer (e.g., bladder transitional cell carcinoma), squamous cell cancer, breast cancer, cervical cancer, or endometrial cancer.

The invention also features, a substantially pure and isolated nucleic acid molecule that includes the nucleic acid sequence of SEQ ID NO: 1.

In one preferred embodiment, the nucleic acid molecule encodes a polypeptide, or fragment thereof, that enhances Wee-1 mediated tyrosine phosphorylation of cdk2. In other preferred embodiments, the nucleic acid molecule is genomic DNA or cDNA. In another embodiment, the nucleic acid molecule is operably linked to a regulatory sequence for expression of the polypeptide, in which the regulatory sequence includes a promoter, for example, a constitutive promoter, a promoter inducible by one or more external agents, or a cell-type specific promoter.

The invention also features a vector containing a nucleic acid molecule that includes the sequence of SEQ ID NO: 1. The vector is preferably capable of directing expression of the polypeptide encoded by the nucleic acid molecule in a vector-containing cell.

The invention also features a transgenic cell, in which the transgene in the cell includes a nucleic acid molecule encoding a Cables polypeptide, and in which the amino acid sequence of the Cables polypeptide includes the sequence of SEQ ID NO: 2. In one embodiment, the nucleic acid molecule is positioned for expression in the transgenic cell. In another embodiment, the transgenic cell is in a transgenic animal.

Another feature of the invention is a substantially pure polypeptide including the amino acid sequence of SEQ ID NO: 2. In one embodiment, this polypeptide, or biologically active fragment thereof, enhances Wee1-mediated tyrosine phosphorylation of cdk2.

The invention also features an antibody that specifically recognizes a human Cables polypeptide.

Finally, the invention features a method of producing a Cables polypeptide, a biologically-active fragment thereof, or an analog thereof by a) providing a cell transformed with a nucleic acid molecule of the invention encoding a Cables polypeptide, or an analog thereof, positioned for expression in the cell, b) culturing the transformed cell under conditions for expressing the nucleic acid molecule, and c) isolating the Cables polypeptide, biologically-active fragment thereof, or analog thereof. In one embodiment, the nucleic acid molecule further includes a promoter inducible by one or more external agents. In another embodiment, the cell is a prokaryotic or eukaryotic cell.

Definitions

By “analog” is meant any substitution, addition, or deletion in the amino acid sequence of a Cables polypeptide that exhibits properties that are at least 30%, preferably at least 50%, more preferably at least 75%, and most preferably at least 95% of the tumor suppressor properties of a Cables polypeptide from which it is derived. Analogs can differ from the naturally occurring Cables protein by amino acid sequence differences, by post-translational modifications, or by both. Analogs of the invention are substantially identical to a naturally occurring Cables sequence. Modifications include in vivo and in vitro chemical derivatization of polypeptides, e.g., acetylation, carboxylation, phosphorylation, or glycosylation. Such modifications may occur during polypeptide synthesis or processing, or following treatment with isolated modifying enzymes. Analogs can also differ from the naturally occurring Cables polypeptide by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989; or Ausubel et al., Current Protocols in Molecular Biology, John Wiley & Sons, New York, 2001. Also included are cyclized peptides, molecules, and analogs that contain residues other than L-amino acids, e.g., D-amino acids or non-naturally occurring or synthetic amino acids.

Fragments and analogs can be generated using standard techniques, for example, solid phase peptide synthesis or polymerase chain reaction.

By “biologically active fragment” is meant a fragment of a Cables polypeptide that possesses any in vivo activity that is characteristic of the full-length Cables polypeptide. A useful biologically active fragment is one which exhibits Cables biological activity in any biological assay described herein, for example, Wee1-mediated cdk2 tyrosine phosphorylation. As used herein, the term “fragment”, as applied to a polypeptide, will ordinarily be at least 20 residues, more typically at least 40, 60, 80, or 100 residues, and preferably at least 150, 200, 250, 300, 350, 400 or more residues in length. Fragments of a Cables polypeptide can be generated by methods known to those skilled in the art. The ability of a candidate fragment to exhibit a biological activity of Cables can be assessed by those methods described herein. Also included in the invention are Cables polypeptides containing residues that are not required for biological activity of the peptide, e.g., those added by alternative mRNA splicing or alternative protein processing events.

By “biological parameter” is meant a measurable characteristic that results from a biological process, such as the level of transcription of a Cables gene or translation of a Cables mRNA, the biological activity of a Cables polypeptide, or the amount of Cables mRNA or polypeptide induced by a stimulus.

By “Cables biological activity” or “Cables activity” is meant the ability to regulate the cell cycle and preferably to promote Wee1-mediated cdk2 tyrosine phosphorylation. Preferably, a polypeptide according to the invention has at least 5%, 10%, 20%, 40%, 50%, 75%, 90%, 95%, or even 100% of the Cables biological activity exhibited by a human wild-type Cables polypeptide that includes the sequence of SEQ ID NO: 2, under identical assay conditions. A protein having “Cables biological activity” or “Cables activity” may also interact with other endogenous cellular proteins. Interactions with these endogenous cellular proteins may be measured using techniques known to those skilled in the art, for example, by column chromatography or by an immunoprecipitation assay, as well as by western blot using, for example, anti-phosphotyrosine antibodies. In addition, further guidance for assaying protein interactions or function may be found in, for example, Ausubel et al. (supra).

By “Cables nucleic acid molecule” is meant a nucleic acid molecule that encodes a polypeptide that has a Cables biological activity, for example, the ability to enhance Wee1-mediated tyrosine phosphorylation of cdk2. Preferably, a “Cables nucleic acid molecule” includes a sequence that is substantially identical to SEQ ID NO: 1 or encodes a Cables polypeptide that includes an amino acid sequence that is substantially identical to SEQ ID NO: 2.

By “Cables gene” is meant a nucleic acid molecule that encodes a polypeptide that has a Cables biological activity and that includes an operably linked promoter and (optionally) regulatory sequences that modulate Cables expression. Preferably, a Cables gene encodes a Cables polypeptide that includes an amino acid sequence substantially identical to SEQ ID NO: 2, or a portion thereof. By “Cables expression” is meant transcription and/or translation of a “Cables gene.”

By “Cables promoter” is meant a nucleic acid sequence that is normally positioned adjacent to a Cables nucleic acid sequence and regulates Cables gene transcription.

By “promoter” generally is meant the minimal sequence sufficient to direct transcription of an operably linked nucleic acid. The promoter region of a known gene may be readily determined, using standard techniques, by one skilled in the art. Desired promoters for heterologous gene expression include CMV, SV40, MMTV, MoMLV, and TK. Also included in the invention are those promoter elements that are sufficient to render promoter-dependent gene expression cell-type specific, tissue-specific, or inducible by external signals or agents; such elements may be located in the 5′ or 3′ regions of the native gene.

By “regulatory” is meant that the nucleic acid sequences associated with the Cables gene modulate its expression either at the level of transcription or translation.

By “operably linked” is meant that a gene and a regulatory sequence(s) are connected in such a way as to permit gene expression when the appropriate molecules (e.g., transcriptional activator proteins) are bound to the regulatory sequence(s).

By “Cables polypeptide” or a “Cables protein” is meant an amino acid sequence that has a Cables biological activity. Preferably, a “Cables polypeptide” or “Cables protein” has at least 5%, 10%, 20%, 40%, 50%, 75%, 90%, 95%, or even 100% of the activity of a human wild-type Cables polypeptide that includes the amino acid sequence of SEQ ID NO: 2.

By “substantially pure and isolated nucleic acid molecule” is meant a nucleic acid molecule that is free of the genes which, in the naturally occurring genome of the organism from which the nucleic acid molecule of the invention is derived, flank the gene. The term therefore includes, for example, a recombinant nucleic acid molecule that is incorporated into a vector; into an autonomously replicating plasmid or virus; or into the genomic DNA of a eukaryote or prokaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding additional polypeptide sequence.

By “substantially pure polypeptide” is meant a Cables polypeptide which has been separated from components that naturally accompany it. Typically, the polypeptide is substantially pure when it is at least 60%, by weight, free from the proteins and naturally-occurring organic molecules with which it is naturally associated. It is desirable for the preparation to be at least 75%, more desirably at least 90%, and even more desirably 95%, and most desirably 99%, by weight Cables polypeptide. A substantially pure polypeptide may be obtained, for example, by extraction from a natural source (e.g., a human cell); by expression of a recombinant nucleic acid molecule encoding the polypeptide in a host cell that does not naturally produce the polypeptide; or by chemically synthesizing the protein. Purification of polypeptides may be by techniques known in the art, such as those described in Ausubel et al., supra, and Methods in Enzymology, and include, for example, immunoprecipitation, differential extraction, salt fractionation, column chromatography, such as on ion exchange resins and immunoaffinity chromatography, magenetic bead immunoaffinity purification, panning with a plate-bound antibody, centrifugation, and the like. Purity can be measured by any appropriate method, e.g., those described in column chromatography, polyacrylamide gel electrophoresis, or by HPLC analysis.

A protein is substantially free of naturally associated components when it is separated from those contaminants which accompany it in its natural state. Thus a protein which is chemically synthesized or produced in a cellular system different from the cell from which it naturally originates will be substantially free from its naturally associated components. Accordingly, substantially pure polypeptides include those derived from eukaryotic organisms, but synthesized in other eukaryotes or prokaryotes.

By “substantially identical” is meant a polypeptide or nucleic acid exhibiting at least 50%, preferably 60%, 70%, 75%, or 80%, more preferably 85%, 90% or 95%, and most preferably 99% identity to a reference amino acid or nucleic acid sequence. For polypeptides, the length of comparison sequences will generally be at least 15 amino acids, preferably at least 20 contiguous amino acids, more preferably at least 25, 50, 75, 90, 100, 150, 200, 250, 300, or 350 contiguous amino acids, and most preferably the full-length amino acid sequence. For nucleic acids, the length of comparison sequences will generally be at least 45 contiguous nucleotides, preferably at least 60 contiguous nucleotides, more preferably at least 75, 150, 225, 275, 300, 450, 600, 750, 900, or 1000 contiguous nucleotides, and most preferably the full-length nucleotide sequence.

Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

Multiple sequences may also be aligned using the Clustal W(1.4) program (produced by Julie D. Thompson and Toby Gibson of the European Molecular Biology Laboratory, Germany and Desmond Higgins of European Bioinformatics Institute, Cambridge, UK) by setting the pairwise alignment mode to “slow,” the pairwise alignment parameters to include an open gap penalty of 10.0 and an extend gap penalty of 0.1, as well as setting the similarity matrix to “blosum.” In addition, the multiple alignment parameters may include an open gap penalty of 10.0, an extend gap penalty of 0.1, as well as setting the similarity matrix to “blosum,” the delay divergent to 40%, and the gap distance to 8.

By “transformed cell” is meant a cell into which (or into an ancestor of which) has been introduced, by means of recombinant DNA techniques, a DNA molecule encoding a polypeptide (for example, a Cables polypeptide).

By “transgene” is meant any nucleic acid molecule that is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “expression vector” is meant a recombinant nucleic acid molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

By “positioned for expression” is meant that the nucleic acid molecule is positioned adjacent to a nucleic acid sequence that directs transcription and translation of the sequence (i.e., facilitates the production of, e.g., a Cables polypeptide, a recombinant polypeptide, or an RNA molecule).

By “modulate” or “modulation” is meant to either increase or decrease the activity of a polypeptide relative to that observed under control conditions. For example, Cables biological activity may be measured by determining the level of Wee1-mediated cdk2 tyrosine phosphorylation, or by the level of expression of a Cables mRNA or polypeptide, or a reporter construct that is under the control of a Cables-specific promoter. The modulation in Cables biological activity is desirably an increase or decrease of at least 5%, 10%, 20%, 40%, 50%, 75%, 90%, 100%, 200%, 500%, or even 1000%.

By “level of expression” of “expression” is meant the amount of transcription or translation of a specific gene, for example, a Cables gene, which can be measured. A change in the level of expression may be determined, for example, for a protein or nucleic acid molecule, and may be either an increase or a decrease relative to the level of a protein or nucleic acid molecule under control conditions. The change in the level of expression is desirably an increase or decrease of at least 5%, 10%, 20%, 40%, 50%, 75%, 90%, 100%, 200%, 500%, or even 1000%.

By “reporter construct” is meant a nucleic acid molecule engineered to include a promoter (e.g., a Cables promoter) that will drive expression of a gene whose expression may be assayed; such genes include, without limitation, those encoding glucuronidase (GUS), luciferase, chloramphenicol transacetylase (CAT), green fluorescent protein (GFP), alkaline phosphatase, and β-galactosidase.

By “candidate compound” or “test compound” is meant a chemical, be it naturally-occurring or artificially-derived, that is surveyed for its ability to modulate Cables biological activity, for example, in one of the assay methods described herein. Candidate compounds may include, for example, nucleic acid molecules, peptides, polypeptides, synthetic and recombinant pharmaceuticals, synthetic organic molecules, naturally-occurring organic molecules, compound analogs, hormones, antimicrobials, antibiotics, and components thereof. The term may refer to any medicinal substance used in humans or other animals.

By “naturally occurring,” as applied to an object, is meant that an object can be found in nature. For example, a polypeptide or polynucleotide sequence that is present in an organism (including viruses) that can be isolated from a source in nature and which has not been intentionally modified by man in the laboratory is naturally occurring.

By “specifically recognizes,” as used herein in reference to an antibody, is meant an increased affinity of an antibody for the protein against which it was raised, relative to an equal amount of any other protein. For example, an anti-Cables antibody desirably has an affinity for Cables that is least 2-fold, 5-fold, 10-fold, 30-fold, or 100-fold greater than for an equal amount of any other protein.

By “mutation” or “alteration” is meant a change in a naturally-occurring or reference nucleic acid sequence, such as an insertion, deletion, frameshift mutation, nonsense mutation, or missense mutation. Desirably, the amino acid sequence encoded by the nucleic acid sequence has at least one amino acid alteration from a naturally-occurring sequence.

By “cancer susceptibility gene,” is meant any gene that, when altered, increases the likelihood that the organism carrying the gene will develop a cancer-related condition during its lifetime. Examples of such genes include proto-oncogenes, tumor suppressor genes, and genes involved in the regulation of cell growth, the cell cycle, and apoptosis.

By “cancer-related condition” is meant any disorder characterized by abnormal cell proliferation, as well as diseases and conditions that directly relate to such abnormal cell growth. Specific examples of cancer-related conditions include various types of cancer, for example, prostate cancer, ovarian cancer, colorectal cancer (e.g., colorectal adenocarcinoma), stomach cancer, breast cancer, lung cancer, esophageal cancer, head cancer, neck cancer, bladder cancer (e.g., bladder transitional cell carcinoma), squamous cell cancer, pancreatic cancer, cervical cancer, and endometrial cancer.

By “abnormal proliferation,” is meant uncontrolled or disregulated division by a cell, for example, division by a cell that normally does not undergo cell division. Abnormal proliferation includes cell division that is abnormally rapid or that occurs at an abnormal time or place in a subject.

By “proliferative disease-associated alteration” or “cancer-associated alteration,” is meant any genetic change within a differentiated cell that results in the abnormal proliferation of that cell. Desirably, such a genetic change correlates with a statistically significant (p is less than or equal to 0.05) increase in the risk of developing a tumor, cancer, or other proliferative disease. Examples of such genetic changes include mutations in genes involved in the regulation of the cell cycle, of growth control, or of apoptosis and can further include mutations in tumor suppressor genes and proto-oncogenes.

By “genetic lesion,” is meant a nucleic acid change. Examples of such a change include single nucleic acid changes as well as deletions and insertions of one or more nucleic acids, duplications, and inversions. A genetic lesion may be a naturally-occurring polymorphism, for example, one that predisposes an animal carrying the polymorphism to develop a cancer-related condition, to have a different treatment prognosis, or to respond differently to therapeutic treatment.

By “treating, stabilizing, or preventing cancer” is meant causing a reduction in the size of a tumor or number of cancer cells, slowing or preventing an increase in the size of a tumor or cancer cell proliferation, increasing the disease-free survival time between the disappearance of a tumor or other cancer and its reappearance, preventing an initial or subsequent occurrence of a tumor or other cancer, or reducing an adverse symptom associated with a tumor or other cancer. In one desired embodiment, the percent of tumor or cancerous cells surviving the treatment is at least 20, 40, 60, 80, or 100% lower than the initial number of tumor or cancerous cells, as measured using any standard assay, such as those described herein. Desirably, the decrease in the number of tumor or cancerous cells induced by administration of a compound of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or number of cancerous cells as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after at least 5, 10, 15, or 20 years.

By “hypermethylation” is meant a greater than normal occurrence of covalent modification of cellular substrates with methyl groups, for example, cytosine in CpG islands.

By “CpG island” is meant a region of genomic nucleic acid sequence that is unusually rich in cytosine and guanine nucleotides. The CpG refers to a C nucleotide immediately followed by a G. The “p” in “CpG” refers to the phosphate group linking the two bases. Detection of regions of genomic sequences that are rich in the CpG pattern is important because such regions are resistant to methylation and tend to be associated with genes which are frequently switched on. In the last few years, numerous studies have dealt with the hypermethylation of CpG islands in promoters that is frequently observed in cancer cell lines. Promoter hypermethylation is capable of repressing the expression of genes, a process which may contribute to carcinogenesis especially if the promoters of tumor suppressor genes are affected.

### BRIEF DESCRIPTION OF THE DRAWINGS

The drawings will first be described.

Drawings

FIGS. 1A and 1B are images showing COS7 cells (FIG. 1A) and human (Wi38) fibroblasts (FIG. 1B) transfected with Cables that were stained with affinity purified anti-Cables antibody. Cables is predominantly detected in the nucleus of proliferating cells with less membrane and cytoplasmic staining.

FIG. 2A is an image of a western blot of HeLa Cables and control cell lysates (50 μg) that was probed with anti-Cables antisera.

FIG. 2B is an image of a western blot of cyclin A, cdk2, and p21 after immunoprecipitation with antibodies to cdk2 and immunoprecipitation with antibodies to Cables from HeLa lysates. The western blot analysis for Cyclin E (data not shown) also showed equivalent levels in both cdk2 and Cables lanes.

FIG. 2C is an image of a western blot of cdk2 and tyrosine phosphorylated cdk2. COS7 cells were transfected with cdk2 and Cables, Wee1, or both, and cell lysates were subjected to cdk2 immunoprecipitation followed by antiphosphotyrosine western blot. In the lower panel, the lysates were reprobed with antibody to cdk2. Numbers indicate fold induction over the basal level.

FIG. 2D is an image of a chemiluminescent assay in which immunoprecipitations of cdk2 from Cables and control cell lysates were probed with an antibody specific for Y15-phosphorylated cdk2 and cdk2.

FIG. 2E is an image of a radioimmunoassay in which Cables and control cell lysates were subjected to cdk2 immunoprecipitation followed by in vitro [γ-³²P]ATP kinase assays using histone H1 as a substrate. Equivalent levels of cdk2 were immunoprecipitated, as indicated in FIG. 2D.

FIG. 3 is an image of a western blot of cdk2 and tyrosine phosphorylated cdk2. COS7 cells were transfected with cdk2 and Cables, Wee1, c-Ab1, or all three, and cell lysates were subjected to cdk2 immunoprecipitation followed by antiphosphotyrosine western blot. In the lower panel, the lysates were reprobed with antibody to cdk2.

FIG. 4 is a graph showing growth of stable HeLa cell lines with Cables or control vector. The doubling time of Cables cell lines (at the exponential growth phase) was twice that of the control cell lines.

FIG. 5A is an image of a fluorescent signal that is detected during FISH mapping of the Cables gene on chromosome 18.

FIG. 5B is an image of fluorescent DAPI staining of human chromosomes. The same mitotic figure stained in FIG. 5A, is shown stained with DAPI to identify human chromosome 18.

FIG. 5C is an illustration showing the detailed mapping results from 10 high quality photographs of chromosome 18.

FIGS. 6A and 6B are images showing the expression of Cables in multiple human tumors (Clontech). Cables mRNA was PCR-generated using cDNA derived from multiple human tumors as a PCR template and Cables-specific primers; PCR #1 (FIG. 6A) and PCR #2 (FIG. 6B). Cables cDNA (Lane 1) was used as a positive control followed by negative control (Lane 2). Cables mRNA was decreased in one of two colon cancers, but not in the other tumors (breast, lung, and pancreas shown).

FIG. 6C is an image showing the expression of G3PDH in multiple human tumors (Clontech). As a control, G3PDH mRNA was PCR-generated, using cDNA derived from multiple human tumors as a PCR template and G3PDH-specific primers.

FIGS. 7A, 7B, 7C, and 7D are images showing that Cables is present in normal breast tissue (FIG. 7A), colon tissue (FIG. 7B), pancreas tissue (FIG. 7C), and squamous epithelial tissue (FIG. 7D) by immunohistochemical staining of normal tissue with affinity-purified Cables antisera. Normal breast, colon, pancreas, and squamous epithelium show strong nuclear Cables staining.

FIGS. 7E, 7F, 7G, and 7H are images showing that Cables is present in invasive breast (FIG. 7E) and pancreas (FIG. 7G) carcinomas as determined by strong nuclear staining, but is lost in approximately one-half of invasive colon (FIG. 7F) and squamous (FIG. 7H) cell cancers. The results were obtained by immunohistochemical staining of tumors with affinity-purified Cables antisera.

FIGS. 8A and 8B are images of representative examples of tumor-specific allelic loss on chromosome 18q. Both cases are informative. This figure shows colon cancers with LOH (FIG. 8A) and without LOH (FIG. 8B) of 18q11-12, using microsatellite marker D18S44. T, tumor sample; N, paired normal control. Arrow, the lost allele in the tumor sample.

FIG. 9 is a diagram that illustrates the possible role of Cables in growth inhibition and development. In response to growth inhibitory and developmental signals, Cables enhances cdk2 Y15 phosphorylation by Wee1, an inhibitory phosphorylation, which leads to decreased cdk2 activity and growth inhibition. Cables also enhances cdk5 Y15 phosphorylation by activated c-Ab1, which leads to increased cdk5 activity and is critical for proper neuronal development. Cables expression in normal epithelium suggests that Cables may also play a role in epithelial cell development. Loss of Cables in some colon and squamous cancers suggests that Cables may be involved in the pathogenesis of these tumors.

FIG. 10 is the nucleotide (SEQ ID NO: 1) and amino acid (SEQ ID NO:2) sequences of human Cables.

FIGS. 11A and 11B are photographs of endometrial tissue during the proliferative phase (FIG. 11A) and the secretory phase (FIG. 11B) of the menstrual cycle demonstrating that Cables expression is hormonally regulated. Cables expression is down-regulated by estrogen (FIG. 11A) and up-regulated by progesterone (FIG. 11B).

FIGS. 12A and 12B are photographs showing the loss of Cables nuclear staining indicative of loss of Cables expression in hyperplastic endometrial cells. FIG. 12A (inset) is a photograph showing an enlarged section of FIG. 12A clearly demonstrating that Cables expression is completely absent. FIG. 12B is a photograph showing Cables expression in normal secretory endometrium.

FIGS. 13A and 13B are photographs showing the loss of Cables nuclear staining indicative of loss of Cables expression in endometrial adenocarcinoma. FIG. 13A (inset) is a photograph showing an enlarged section of FIG. 13A clearly demonstrating that Cables expression is completely absent. FIG. 13B is a photograph showing Cables expression in normal secretory endometrium.

FIG. 14 is an image of a western blot showing loss of Cables expression in grade 2 and grade 3 endometrial adenocarcinomas versus normal secretory endometrium.

FIGS. 15A and 15B are images showing northern analysis of Cables mRNA levels in HES cells (FIG. 15A) and SK-UT2 cells (FIG. 15B) following progesterone treatment.

FIGS. 16A and 16B are images showing northern analysis of Cables mRNA levels in HES cells (FIG. 16A) and SK-UT2 cells (FIG. 16B) following progesterone or estrogen treatment.

FIG. 17 is a graph showing that Cables expression slows the proliferation rate of HES cells.

FIG. 18 is a graph showing that Cables expression has no effect on the growth curve of SK-UT2 cells.

FIGS. 19A and 19B are photographs comparing the morphology of wild-type mouse endometrium (FIG. 19A) to Cables-deficient mouse endometrium (FIG. 19B) following gonadotropin stimulation which synchronizes the endometrial tissue at the proliferative state. The epithelium of the endometrium of the wt mice appears uniform, while the epithelium of the endometrium of Cables-deficient mice is hyperplastic, abnormally large and distended, and disorganized.

FIGS. 20A, 20B, 20C, and 20D are additional photographs comparing the difference between wild-type mouse endometrium (FIGS. 20A and 20B) and Cables-deficient mouse endometrium (FIGS. 20C and 20D) following gonadotropin stimulation.

FIGS. 21A, 21B, and 21C are photographs showing immunohistochemical staining of Cables in normal ovary. Strong nuclear staining was seen in ovarian surface epithelium (FIG. 21A), ovarian stromal cells (FIG. 21B), and fallopian tube epithelium (FIG. 21C).

FIGS. 22A and 22B are photographs showing strong immunohistochemical staining of Cables in ovarian mucinous carcinoma (FIG. 22A) and clear cell carcinoma (FIG. 22B).

FIGS. 23A and 23B are photographs showing immunohistochemical staining of Cables showing negative Cables expression (FIG. 23A) and positive Cables expression (FIG. 23B) in ovarian serous carcinoma.

FIGS. 24A and 24B are photographs showing immunohistochemical staining of Cables showing negative Cables expression (FIG. 24A) and positive Cables expression (FIG. 24B) in ovarian endometrioid carcinoma.

FIGS. 25A and 25B are fluorescent images of Cables and control G3PDH mRNA levels by RT-PCR. Seven representative tumor samples from human tumor xenografts were analyzed. FIG. 25A: Cables mRNA expression was detected in breast cancer (lane 3), pancreatic carcinoma (lane 7), one lung cancer (lane 4), and one colon cancer (lane 5). Cables mRNA expression was markedly decreased in ovarian cancer (lane 9), one lung cancer (lane 8), and one colon cancer (lane 6). Lane 1 was a positive control with Cables cDNA. FIG. 25B: G3PDH was used as a control for normalization of the mRNA levels in different samples.

FIG. 26 is a graph showing that Cables slows the proliferation rate when expressed in Du-145 cells.

FIG. 27 is a graph demonstrating that Cables expression in HeLa cells results in a greater than 50% reduction in the growth rate.

FIG. 28 is an image of a western blot showing overexpression of Cables in HeLa cells.

FIGS. 29A and 29B are photographs showing the effect of Cables overexpression on tumor formation in nude mice. HeLa cells expressing high or low levels of Cables were administered to nude mice and tumor formation evaluated. Seven out of 9 nude mice administered HeLa cells expressing low amounts of Cables developed tumors (FIG. 29A), while only 2 out of 9 nude mice developed tumors when administered HeLa cells expressing high amounts of Cables (FIG. 29B).

FIG. 30 is a photograph showing the absence of Cables expression in tumors formed from parental HeLa cells (not transfected with Cables) administered to nude mice. Inset A demonstrates the lack of Cables in the tumor tissue, while inset B demonstrates the presence of Cables expression in the surrounding liver tissue.

FIGS. 31A and 31B are photographs showing that Cables overexpression in HES cells following infection with a Cables adenoviral construct (Ad-Cables; FIG. 31A) inhibits cellular proliferation, while a control vector has no effect (FIG. 31B).

### DETAILED DESCRIPTION

The presently claimed invention is based on the discovery that the cdk-interacting polypeptide Cables is predominantly located in the nucleus of proliferating cells and expression of Cables in actively growing cells inhibited cell growth. Cables enhanced cdk2 tyrosine 15 (Y15) phosphorylation by Wee1, an inhibitory phosphorylation, and caused decreased cdk2 kinase activity. The Cables gene is located on chromosome 18q11-12, a site of allelic loss in many human cancers. We found lack of Cables protein expression in 50-60% of primary human colon and head and neck squamous cell carcinomas, and in greater than 70% of ovarian serous tumors. Cables expression has also been evaluated in normal, hyperplastic, and malignant human endometrium. Analysis of endometrial tissue samples, as well as endometrial cell lines, indicates that loss of Cables protein is associated with the transition from normal to hyperplastic endometrium. In all endometrial cancers examined, the Cables protein was absent. In addition, tumors generated in nude mice from cultured cells that expressed Cables mRNA failed to express a functional protein. The data indicate that loss of Cables and inhibition of cyclin E/cdk2 tyrosine phosphorylation are critical in cell growth regulation and the pathogenesis of human cancers (e.g., the malignant transformation of the endometrium).

Cables mRNA expression is also hormonally regulated. Progesterone can dose dependently elevate Cables mRNA expression, which acts to slow the cell cycle, while estrogen reduces the level of Cables mRNA expression. Both hormones play an important role in regulating cancer development.

Cables Is Predominantly a Nuclear Protein

Cables cDNA encodes a protein of 568 amino acid residues with a predicted molecular size Of Mᵣ 63,000. Cables displays little sequence homology to other known proteins in the databases. It does, however, show weak homology to cyclin A and weaker homology to cyclin C over an ˜200 amino acid stretch in the C-terminal third of the protein that may be the cdk-interacting region. Cables contains two tyrosine-based sorting motifs (YXXLE), which have been implicated in axonal growth cone sorting and explain its presence in the axonal growth cone in mature neurons (Zukerberg et al., Neuron 26:633-646, 2000). There are three classic nuclear localization signals (Hicks and Ratikhel, Annu. Rev. Cell Dev. Biol. 11:155-188, 1995) composed of 3 basic amino acids, and either histidine or proline at amino acid positions 48, 54, and 406. These nuclear signals exhibit both pattern 4 (4 residue) and pattern 7 (starting with P and followed within 3 residues by a basic segment). Another bipartite nuclear localization signal is located at position 264 (Robbins et al., Cell 64: 615-623, 1991). Moreover, a cytoplasmic/nuclear discrimination score based on amino acid composition predicts that Cables is a nuclear protein with a 94.1% reliability score (Reinhardt and Hubbard, Nucleic Acids Res. 36:2230-2236, 1998). It is also likely that there are at least two splice variants of Cables.

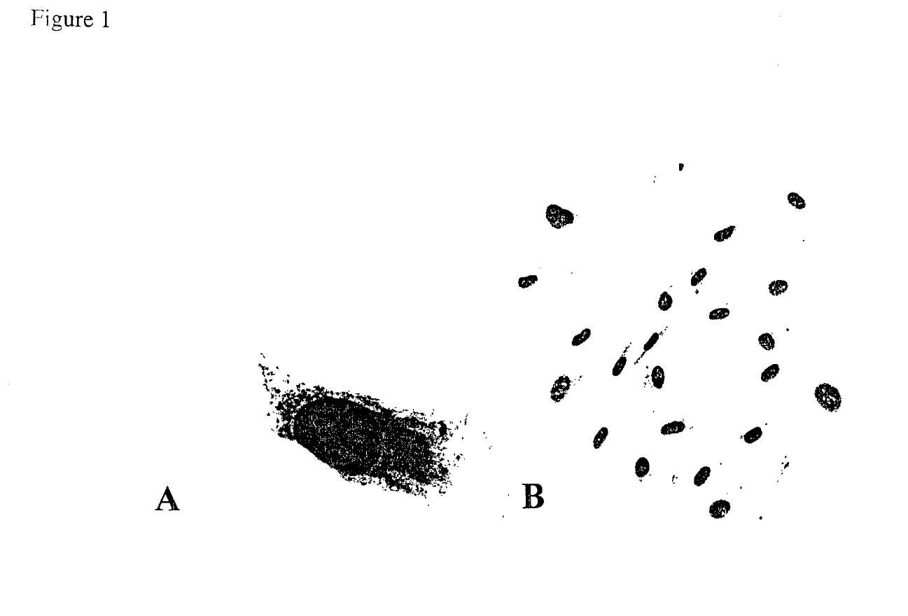

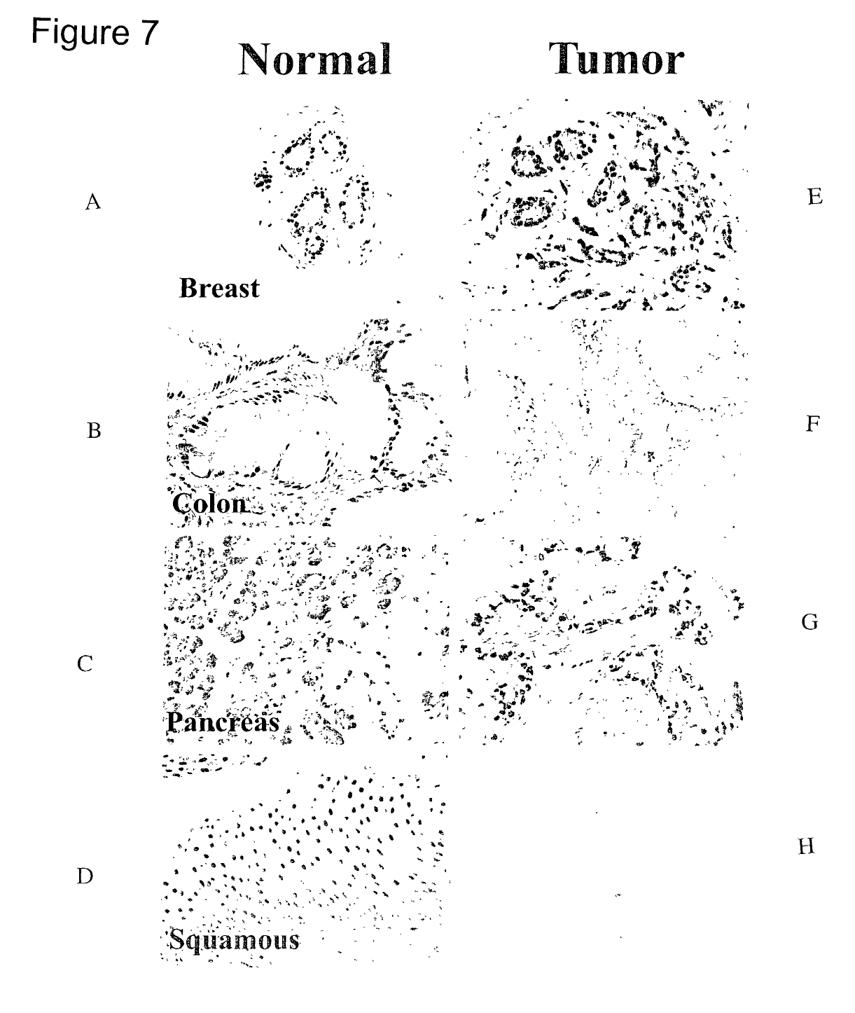

To examine Cables localization in proliferating cells, Cables immunohistochemistry was performed on cell cultures and tissue sections using affinity-purified anti-human Cables antisera (FIGS. 1 and 7). Preabsorbed antisera was used as a control to show specificity. Diffuse nuclear staining was seen in both COS7 cells transfected with Cables and human fibroblasts (FIG. 1). COS7 cells contain little to no endogenous Cables by western blot and untransfected cells showed no Cables staining with the affinity-purified anti-Cables antisera (FIG. 1A). Similarly, diffuse strong nuclear staining was seen in normal squamous and glandular epithelium using paraffin embedded tissue sections and a microwave enhanced staining method (FIG. 7). Cell fractionation studies confirmed the immunohistochemical staining results seen in proliferating cells and brain tissue. In proliferating cells (HeLa and CEM) Cables was predominantly (90%) localized to the nuclear fraction with only small amounts in the cytoplasmic and membrane fractions. In contrast, fractionation studies of adult mouse brain showed that 60-70% of Cables was present in the nuclear fraction and approximately 30% was in the membrane fraction. Thus Cables is likely to be predominantly a nuclear protein in proliferating cells, but also is located at axonal growth cones where it appears to have a specific function in neurite outgrowth.

Cables Inhibits Cell Growth

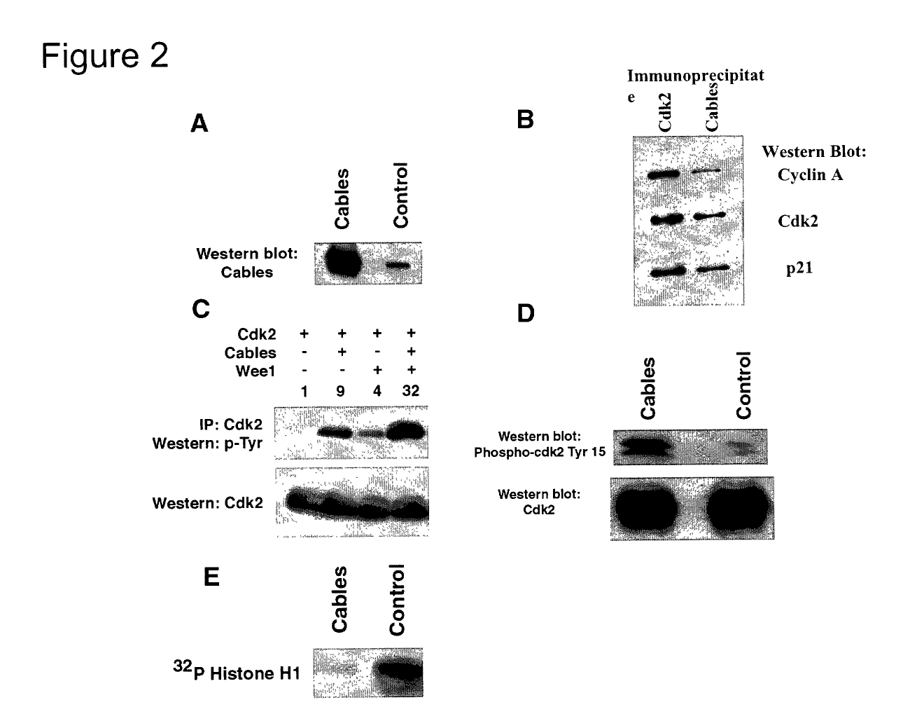

In proliferating cells Cables is a nuclear protein that interacts with multiple cdks, including cdk2 and cdk3 (Zukerberg et al., Neuron 26:633-646, 2000; Matsuoka et al., Biochem Biophys. Res. Commun. 273:442-447, 2000). To examine a role of Cables in cell growth, HeLa cell lines stably expressing Cables were generated. The pCIN4 vector was used to ensure that all antibiotic-resistant cells expressed the recombinant protein (Rees et al., Biotechniques 20:102-110, 1996). Two stable cell lines were generated that showed an increased amount of Cables protein by both western blot (FIG. 2A) and immunostaining. Three control lines (vector alone) were also generated and compared to the Cables cell lines. Both Cables cell lines showed approximately 10 fold more Cables than endogenous levels found in the control HeLa cells and immunohistochemical staining showed diffuse nuclear staining in both the control and Cables cell lines. Growth characteristics of the three control and two Cables cell lines were studied (Table 1); each experiment was performed three times using all five cell lines and the range of values for all experiments is given.

TABLE 1
Growth characteristics of cables and control cell lines

| | Control | Cables |
|---|---|---|
| Doubling time | 9-12 h | 24-28 h |
| Thymidine uptake | 100% | 62-68% |
| Colony formation | 47-58% | 16-20% |
| Seeding efficiency | 81-85% | 82-86% |
| Soft agar growth | 1 | 0.4-0.6 |

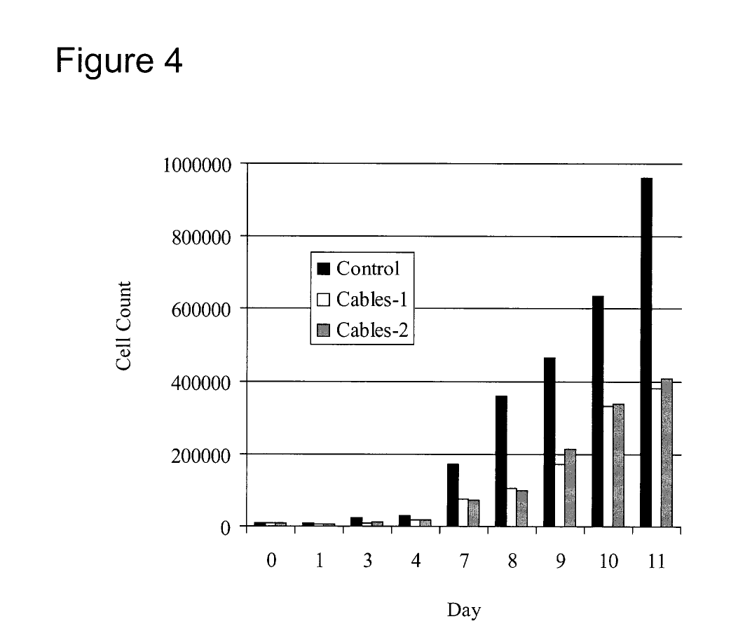

The growth rate of the cell lines was determined by seeding 1×10⁴ cells/well and counting the cells for up to 13 days. Increased numbers of control cells were present after 24 hours and throughout the experiment (FIG. 4). The doubling time in the exponential growth phase for the control cells was calculated to be between 9-12 hours (ATCC reports similar doubling time for HeLa cells) and between 24-28 hours for the cells over-expressing Cables. Differences in growth rates should be accompanied by differences in the rate of DNA synthesis. [³H]thymidine was added to identical numbers of cells and uptake measured after 30 minutes, 1 hour, and 2 hours. The Cables cell lines showed a 35% (range 31-38) decrease in [³H]thymidine uptake compared to control cells at each time point. In addition, the Cables cell lines showed reduced colony formation (a measure of both seeding efficiency and growth) but equal seeding efficiency. Approximately 55% (range 47-58) of the control cells formed colonies compared to 18% (range 16-20) of the Cables cells, despite approximately 80-85% seeding efficiency for both groups. The cell cycle profile of the control and Cables cell lines was analyzed by flow cytometry and found to be similar. No consistent difference in the percentage of cells in the G1-phase, S-phase, or G2/M-phase of the cell cycle was noted between Cables and control cell lines. Similarly no change in the cell cycle profile was seen in Saos2 and HeLa cells transiently transfected with either Cables or a control vector. Terminal deoxynucleotidyl transferase-mediated nick end labeling (TUNEL) assays, as well as careful observation of cell cultures, showed no increase in apoptosis or dead cells in the Cables versus control cell lines. These data suggest that changes in doubling time and growth rate are due to lengthening of multiple phases of the cell cycle rather than due to a cell cycle block or cell death.

Cables Enhances Y15 Phosphorylation of Cdk2 by the Wee1 Kinase

Cables associates with cdk2 (FIG. 2B) and the C-terminal 200 amino acids show weak homology to cyclin A and cyclin C. To examine if Cables growth inhibition was a result of binding to cdk2 and blocking cyclin binding, Cables associated proteins were studied. Upon cotransfection, association of Cables and cdk2, cyclin A, cyclin E, and p21 could be readily demonstrated. No association was seen with cdc2 (cdk1) and p27. Equivalent levels of cdk2, immunoprecipitated directly or through Cables from HeLa cell lysates showed approximately equivalent levels of cyclin A, cyclin E and p21 (FIG. 2B), suggesting that Cables is present in a multimolecular complex with cdk2, cyclin A/E, and p21.

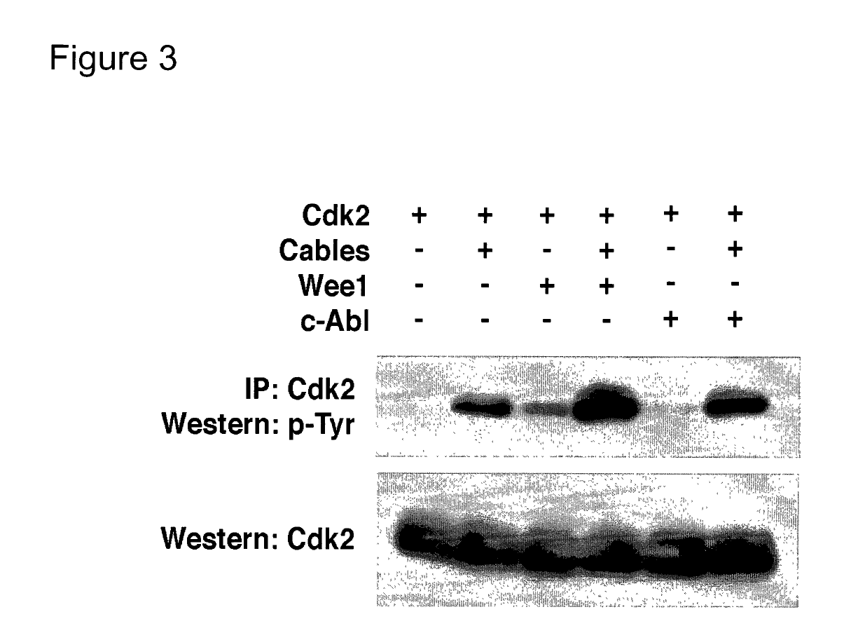

The observation that Cables linked cdk5 and c-Ab1 led us to look at cdk2 tyrosine phosphorylation. Interestingly, cdk2 became tyrosine phosphorylated when Cables or Wee1 was overexpressed, and the level of tyrosine phosphorylation increased exponentially (approximately 30 times basal level) when both Cables and Wee1 were coexpressed (FIGS. 2C and 3). Phosphorylation of the Y15 residue is a known regulatory event for the cdks (1). This residue is conserved in cdk2. A Y15 to phenylalanine cdk2 mutant (F15) was no longer phosphorylated by Cables and Wee1, demonstrating that Cables enhanced tyrosine phosphorylation of cdk2 occurs on the highly conserved Y15 residue. To further verify that Cables enhanced cdk2 Y15 phosphorylation, cdk2 tyrosine phosphorylation was examined in the stable Cables and control cell lines, using a phospho-tyrosine cdc2 (Tyr15) antibody that is specific for Y15-phosphorylated cdc2 and cdk2 (FIG. 2D). The Cables cell lines showed a 5-fold increase in Y15-phosphorylated cdk2 compared to the control cell lines, while the total amount of cdk2 immunoprecipitated from the cells was the same (FIG. 2D). Both Cables overexpressing cell lines showed similar increased levels of Y15-phosphorylated cdk2 (and decreased kinase activity). Y15-phosphorylation of cdc2 and cdk2 by the Wee1 family kinases is inhibitory and must be relieved by the cdc25 family of phosphatases for kinase activation. As expected, we found that cdk2 immunoprecipitated from the Cables cell lines had significantly decreased levels of histone H1 kinase activity than from the control cell lines (FIG. 2E). Similarly, in cells transfected with cdk2, Cables and Wee1, comparable levels of Cables associated cdk2 had much less histone H1 kinase activity than cdk2 that was directly immunoprecipitated from the cell extracts.

Cables Is Not Expressed in One-half of Head and Neck Squamous Cell and Colon Cancers

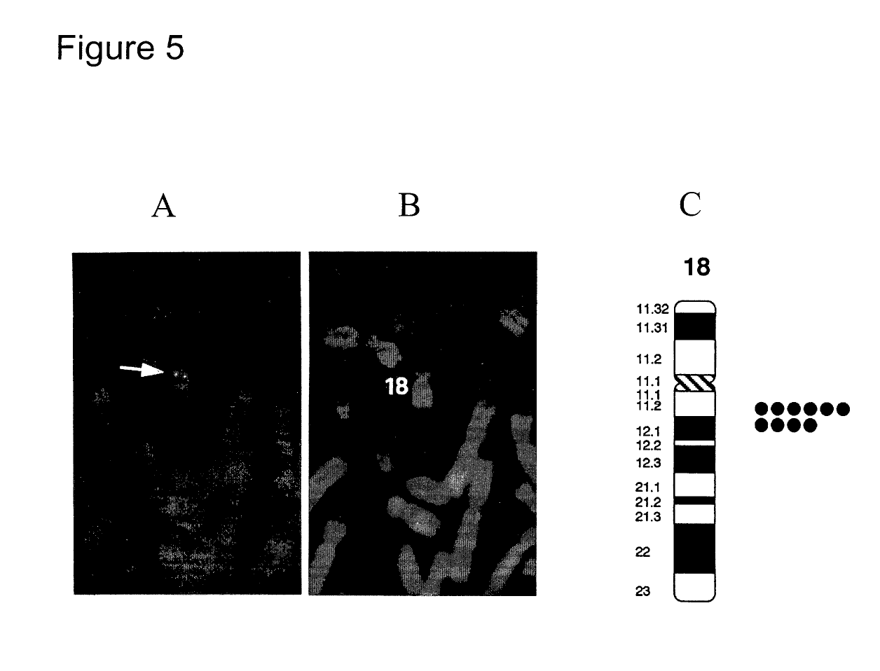

The human Cables cDNA was isolated from a human fetal brain cDNA library using the mouse Cables cDNA (GenBank accession no. AF348525) and was used as a probe for FISH analysis. Cables was found to lie on human chromosome 18, region q11.2-q12.1 (FIG. 5). A search of the genome databases revealed one BAC clone of human chromosome 18 (AC021244) that matched the human Cables sequence, confirming Cables location on human chromosome 18.

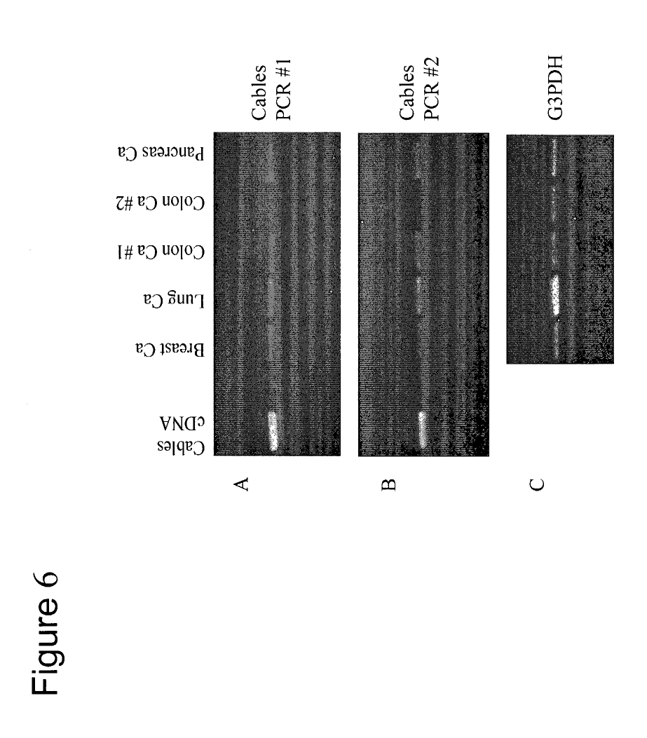

Chromosome 18q abnormalities are found in many human tumors, especially colorectal and pancreatic adenocarcinomas and head and neck squamous cell carcinomas (Thiagalingam et al., Nat. Genet. 13:343-346, 1996; Hoglund et al., Br. J. Cancer 77:1893-1899, 1998; Jones et al., Arch. Otolaryngol. Head Neck Surg. 123:610-614, 1997). Expression of Cables in actively growing cells inhibited cell growth, so we questioned if Cables might be affected in chromosome 18q loss. First-strand cDNA from eight human tumor xenografts (Clontech), including lung, colon, breast, prostate, pancreas and ovary tumors, were screened with two sets of Cables specific primers from the very C-terminal portion of human Cables and G3PDH primers as a control (FIG. 6). Seven tumors showed approximately equal levels of Cables cDNA, however the eighth tumor (one of two colon cancers) showed significantly decreased Cables cDNA with both primer sets. G3PDH cDNA levels were approximately the same in all tumors. These results suggested that Cables may be involved in chromosome 18q loss.

To further study Cables expression in primary human tumors, a specific antibody was raised against a GST-tagged human Cables and affinity-purified. Cables immunohistochemistry was performed on cell cultures and tissue sections using affinity-purified Cables antisera. Adherent cultured cell preparations of COS7 cells, which contain little endogenous Cables by western blot, were transfected with a mammalian Cables expression construct. The untransfected COS7 cells showed little nuclear staining, while the transfected cells showed strong nuclear positivity (FIG. 1A), suggesting the antisera was specific. Paraffin sections of tumors and normal tissue were then stained with the anti-Cables antisera. Preabsorbed antiserum was also used as a negative control. Colon, pancreas, and squamous tumors, which show chromosome 18q loss, as well as breast cancers, which do not often have chromosome 18q abnormalities, were studied (Table 2).

TABLE 2
Cables expression in human tumors

| | Nuclear Cables staining | |
|---|---|---|
| | Positive | Negative |
| Normal breast (20) | 20 | 0 |
| Breast cancer (20) | 20 | 0 |
| Normal colon (20) | 20 | 0 |
| Colon cancer (20) | 9 | 11 |
| Normal pancreas (20) | 20 | 0 |
| Pancreas cancer (20) | 20 | 0 |
| Normal squamous mucosa (20) | 20 | 0 |
| Squamous cell carcinoma (20) | 8 | 12 |

All tumor slides contained adjacent normal tissue on the same slide to serve as an internal control. Normal colon, breast, pancreas and squamous tissue (80 different specimens) showed strong nuclear staining of the squamous and glandular epithelium in all cases (FIG. 7). There was also nuclear staining of the scattered lymphocytes and plasma cells within the lamina propria. Similarly, all cases of breast and pancreas cancer showed strong nuclear staining of the tumor cells. In contrast, 11 of 20 colonic adenocarcinomas (55%) showed lack of nuclear Cables staining, while the other 9 cases showed strong nuclear staining for Cables (Table 2 and FIG. 7). Similarly, 12 of 20 squamous cell carcinomas of the head and neck (60%) showed no Cables staining. All sections contained normal squamous and glandular mucosa, which served as a positive control, and the normal epithelial cells showed strong nuclear staining in all cases, including those in which the tumor was negative. Furthermore, positive staining of lymphocytes and plasma cells present adjacent to negative tumor cells served as an additional internal positive control. Faint cytoplasmic staining was seen in both normal and tumor cells, including many tumors without nuclear staining; the significance of this finding is not clear at this time. These results suggest that loss of Cables is involved in the pathogenesis of some human cancers with chromosome 18q loss. Furthermore lack of Cables expression is specific to colon and squamous cancers as all cases of breast cancer, which do not often have 18q loss, and pancreas cancer, which has been found to have 18q loss in most cases, showed strong nuclear staining for Cables.

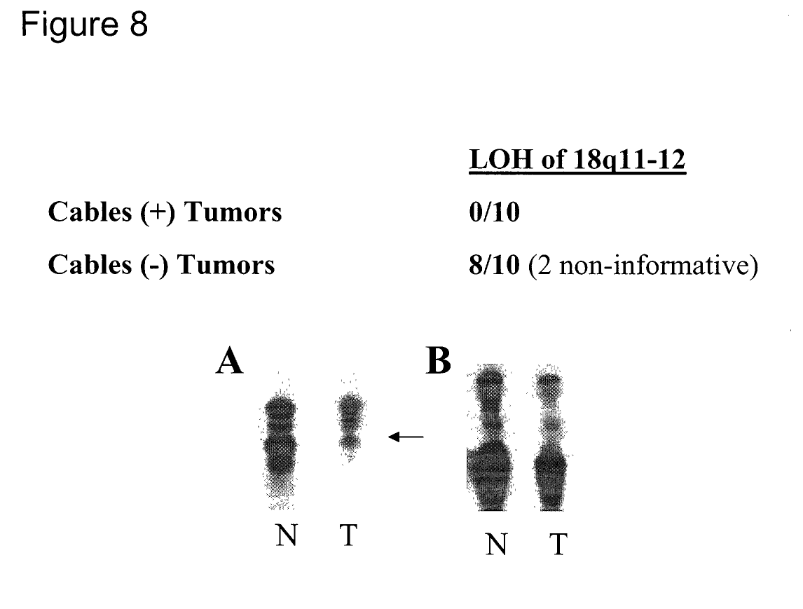

To examine the correlation between Cables expression and loss of chromosome 18q11-12, twenty cases of colon and squamous cancer and normal tissue away from the tumor were studied for LOH using highly polymorphic markers that map to 18q11 (D18S44 and D18S1107), in a “blind” manner. These cases were selected on the basis of having areas composed predominantly of tumor with less desmoplastic stroma and inflammation. LOH studies were performed three times on each sample. Sixteen cases were heterozygous with both markers, while two cases were homozygous with D18S44 and heterozygous with D18S107 and two cases were homozygous with D18S1107 and heterozygous with D18S44. Ten cases (5 colon, 5 squamous) with strong Cables staining showed no LOH on 18q11. In contrast, eight of ten cases of (4 colon, 4 squamous) that lacked Cables staining showed definite LOH of 18q11 compared to the paired normal tissue (FIG. 8). Two cases were indeterminate; tumor DNA did not amplify well in one colon cancer and one squamous cancer showed inconsistent results between trials.

Cancer develops from the transformation of normal epithelium, to a dysplastic epithelial lesion, and ultimately to invasive carcinoma (Fearon and Vogelstein, Cell 61:759-767, 1990). In the colon, this progression is accompanied by a number of recently characterized genetic alterations (Chung, D., Gastroenterology 119:854-865, 2000). Inactivation of the adenomatous polyposis (APC) gene marks one of the earliest events in colorectal carcinoma followed by oncogenic K-ras mutations. Later events include inactivation of the tumor-suppressor gene p53 on chromosome 17p and loss of heterozygosity (LOH) on the long arm of chromosome 18 (18q). Chromosome 18q abnormalities are also common in pancreas cancer and squamous cell cancer of the head and neck. FISH mapping of pancreatic cancer showed that all cases had lost at least one copy of chromosome 18q and that most breakpoints mapped to 18q11 (Hoglund et al., Br. J. Cancer 77:1893-1899, 1998). Similarly, loss or deletion of chromosome 18q is one of the most common chromosome abnormalities in squamous cell cancers, occurring in 55% to 65% of tumors (Van Dyke et al., Genes Chromosomes Cancer 9:192-206, 1994). More sensitive studies with loss of heterozygosity (LOH) showed that 75% of squamous cancers showed 18q loss at one or more loci and in up to 66% this involved 18q11 (Jones et al., Arch. Otolarygol. Head Neck Surg. 123:610-614, 1997). Furthermore, 25% of the tumors showed loss of 18q11.1-q12.3 without distal loss of chromosome 18.

Three candidate tumor-suppressor genes, deleted in colon cancer (DCC), Smad4 (DPC4), and Smad2, have been identified in the distal portion of chromosome 18q. DCC was recently shown to be the netrin-1 receptor and bind directly to netrin-1 (Keino-Masu et al., Cell 87:175-185, 1996; Stein et al., Science 291:1976-1982, 2001). Furthermore, DCC is expressed in both normal colonic mucosa and both primary and metastatic colon cancer (Gotley et al., Oncogene 13:787-795, 1996), and DCC null mice do not develop tumors (Fazeli et al., Nature 386:796-904, 1997). The Smad proteins mediate transforming growth factor (TGF)—effects and regulate genes involved in cell cycle control. Biallelic inactivation of Smad4 occurs in greater than 60% of pancreas tumors with few mutations identified in the Smad genes in squamous and colon cancers (Eppert et al., Cell 86:543-552, 1996; Hahn et al., Cancer Res. 56:490-494, 1996; Schutte et al., Cancer Res. 56:2527-2530, 1996; Takagai et al., Gastroenterology 111: 1369-1372, 1996; Takagi et al., Br. J. Cancer 78:1152-1155, 1998). Thus it is likely that chromosome 18q harbors at least one more proximally located gene that is involved in human cancer. This is supported by the proximal loss of 18q11-12 without distal 18q loss in some tumors.

In this study, we cloned human Cables cDNA, and mapped its genomic location to chromosome 18, region q11.2-12.1. Previous studies of colon cancer specimens found that 38% had lost an entire chromosome 18 and approximately 65% that lost alleles of a subset of markers on chromosome 18q showed LOH in the region that included Cables (Thiagalingam et al., Nat. Genet. 13:343-346, 1996). Similarly up to 66% of squamous cancers showed loss of the region of chromosome 18 that included Cables (Jones et al., Arch. Otolaryngol. Head Neck Surg., 123:610-614, 1997). Thus, we generated antibodies against human Cables to assess Cables expression in tumors. We were able to detect nuclear Cables protein in routinely processed, formalin-fixed paraffin-embedded tissue sections using affinity purified polyclonal antisera. Nuclear staining for Cables was detected in normal squamous and glandular epithelium from the breast, pancreas, colon and head and neck. However, 55% of colon cancers and 60% of squamous cancers showed loss of nuclear Cables staining. In each of these cases normal mucosa was present on the same section and showed strong nuclear staining. In addition, inflammatory cells, which also show nuclear Cables staining, were present in and around the negative carcinoma cells. To examine the specificity of the Cables loss in these cancers, we also studied breast cancers, which do not commonly show chromosome 18q abnormalities (Schenk et al., J. Mol. Med. 74:155-159, 1996) for loss of Cables. All cases showed strong staining without Cables loss. Furthermore, lack of Cables staining was associated with LOH of chromosome 18q11-12 in colon and squamous cancers. Eight of 10 cases with loss of Cables expression showed LOH of chromosome 18q11-12. No LOH was detected in 10 cases of colon and squamous cancer with Cables staining. Only cases which had areas of predominant tumor with less desmoplastic stroma and inflammation were studied for LOH. Even so, in one case tumor DNA did not amplify and one case was inconclusive, possibly due to admixed non-neoplastic cells. In addition, pancreatic carcinomas are known to lose one copy of chromosome 18q in the majority of cases (Hahn et al., Cancer Res. 56:490-494, 1996) and all showed strong Cables staining. Therefore loss of Cables expression is not likely to be a general consequence of 18q LOH and appears to be specific to colon and squamous cancers.

Cables was recently discovered as a cdk interacting protein (Zukerberg et al., Neuron 26:633-646, 2000; Matsuoka et al., Biochem. Biophys. Res. Commun. 273:442-447, 2000). It acts as a link or cable between the cdks and non-receptor tyrosine kinases, such as c-Ab1 (Zukerberg et al., Neuron 26:633-646, 2000). To examine if loss of Cables could affect cell growth, we created stable cell lines with mildly elevated ectopic expression of Cables. Growth of these cells was significantly inhibited compared to the vector control cells, with a decreased doubling time and colony formation rate but identical plating efficiency. The cell cycle profile and degree of apoptosis was similar in both Cables and control cell lines. Inhibition of cell growth by Cables may be related to decreased cdk2 activity. Cdk2 is involved at multiple points in the cell cycle, including the G1/S transition, initiation, and maintenance of DNA replication (S-phase), and entry and progression through mitosis (Hu et al., Mol. Cell. Biol. 21:2755-2766, 2001; Furuno et al., J. Cell. Biol. 147:295-306, 1999), which could explain a normal cell cycle profile despite slower growth. Cables exists in a multiprotein complex with at least cdk2, cyclin A, or cyclin E, and p21, so it does not inhibit cdk2 activity by displacing the cyclin molecule. Recently, an association between PKCeta and cyclin E/cdk2/p21 complex was shown to inhibit cdk2 activity by causing dephosphorylation of cdk2 Thr160 during keratinocyte differentiation. Cables appears to inhibit cdk2 activity by enhancing Wee1 tyrosine 15 phosphorylation of cdk2, which is an inhibitory phosphorylation for the kinase. In transfected cells, Cables and Wee1 act together to dramatically increase cdk2 tyrosine phosphorylation. Despite the functional interaction between Cables and Wee1, we could not demonstrate a stable interaction between these two proteins in transfected cell or endogenous cell lysates, suggesting that the interaction is likely to be more functional than physical. Cables expressing cell lines showed significantly increased levels of Y15 phosphorylated cdk2 and decreased kinase activity.

Loss of Cables could provide a growth advantage to neoplastic cells by allowing faster progression through the cell cycle. Lack of nuclear Cables expression in primary human cancers is frequent in colon and squamous cancers and is likely to be related to 18q LOH plus gene mutations in the remaining gene, leading to an unstable or truncated protein, or hypermethylation of CpG islands in its promoter. The latter hypothesis is attractive, since widespread genomic hypomethylation, which occurs in the setting of localized hypermethylation, has been reported in these cancers (Goelz et al., Science 228:187-190, 1985; Jones and Laird, Nat. Genet. 21:163-167, 1999). Hypermethylation of CpG islands within promoter sequences of specific tumor-suppressor genes can have a potent silencing effect, and has been reported for INK4p16 gene in 28-55% of colon cancers.

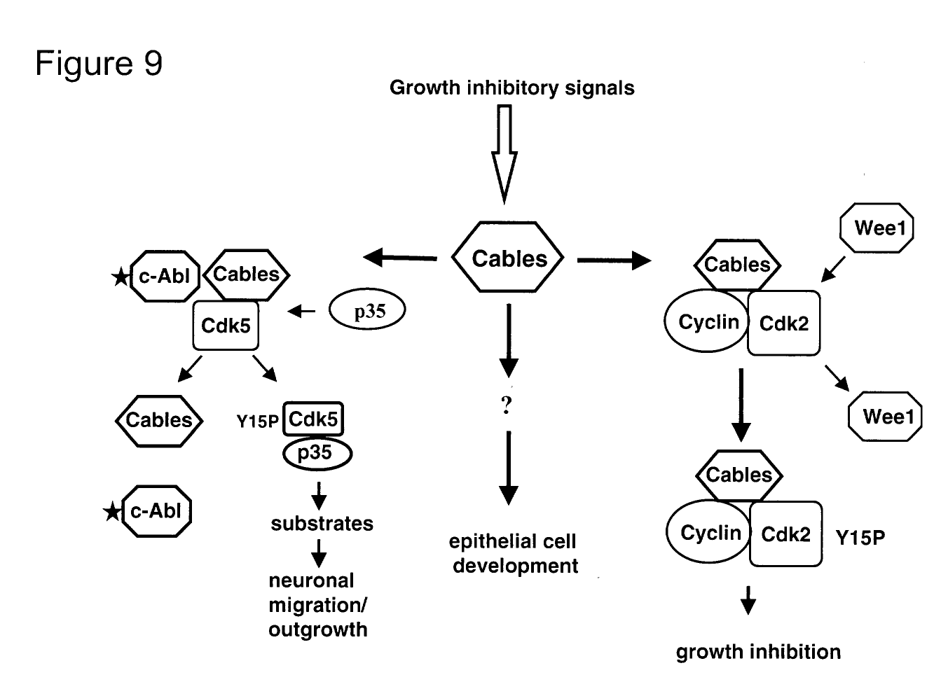

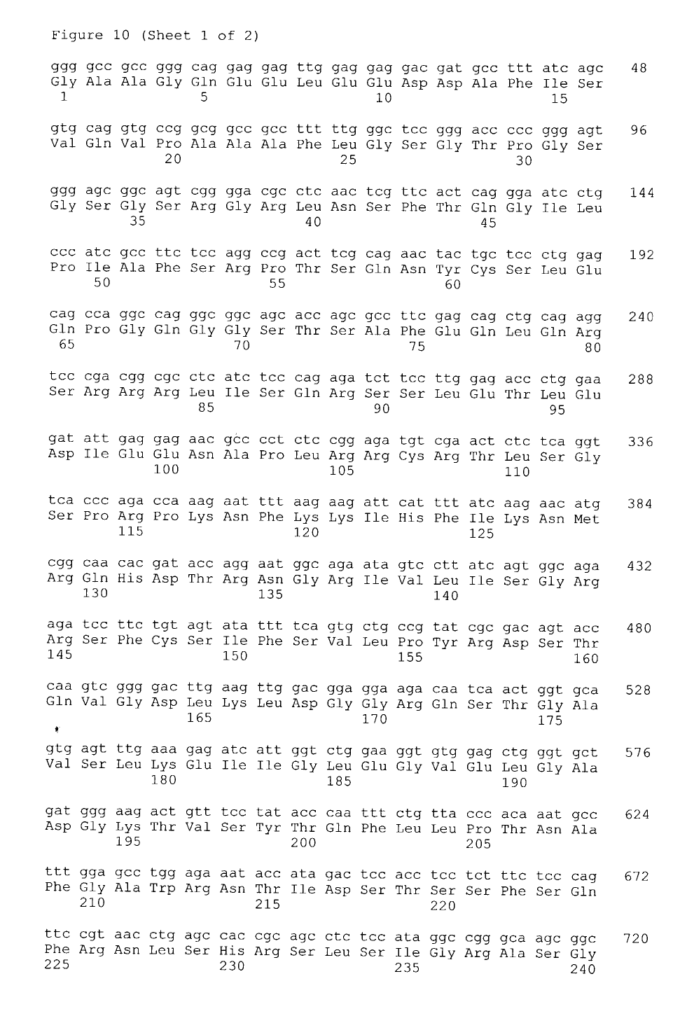

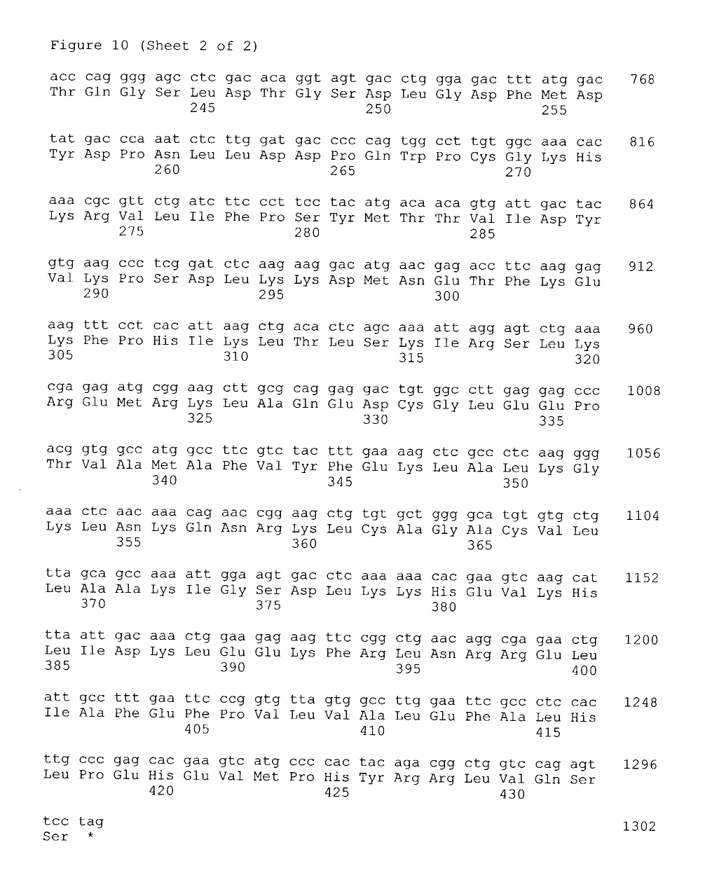

When taken together, our data indicate that Cables is involved in both cell growth and differentiation and, without being bound to a particular theory, indicate that Cables may act as a crossover signal between the two processes (FIG. 9). Cables serves as an adapter molecule that facilitates both cdk5 and cdk2 Y15 phosphorylation, with different consequences. Cdk5Y15 phosphorylation by Cables and c-Ab1, increases kinase activity and contributes to its role in neuronal migration and neurite outgrowth. In primary neuronal cultures, expression of anti-sense Cables caused neurite shortening similar to that seen for dominant-negative cdk5. In contrast, cdk2Y15phosphorylation by Cables and Wee1 is an inhibitory phosphorylation that inhibits cell growth. Furthermore, strong expression of Cables in both normal squamous and glandular epithelium suggests a role for Cables in epithelial development. Loss of Cables may impair growth inhibition and cell differentiation, which can lead to or enhance uncontrolled cell growth and eventually cancer. Lack of expression of Cables in up to 60% of colon and squamous cancers, along with its chromosomal location and role in growth control, indicates that it has a role in the pathogenesis of these tumors.

Loss of Cables Expression Is Associated with Malignant Transformation of the Endometrium

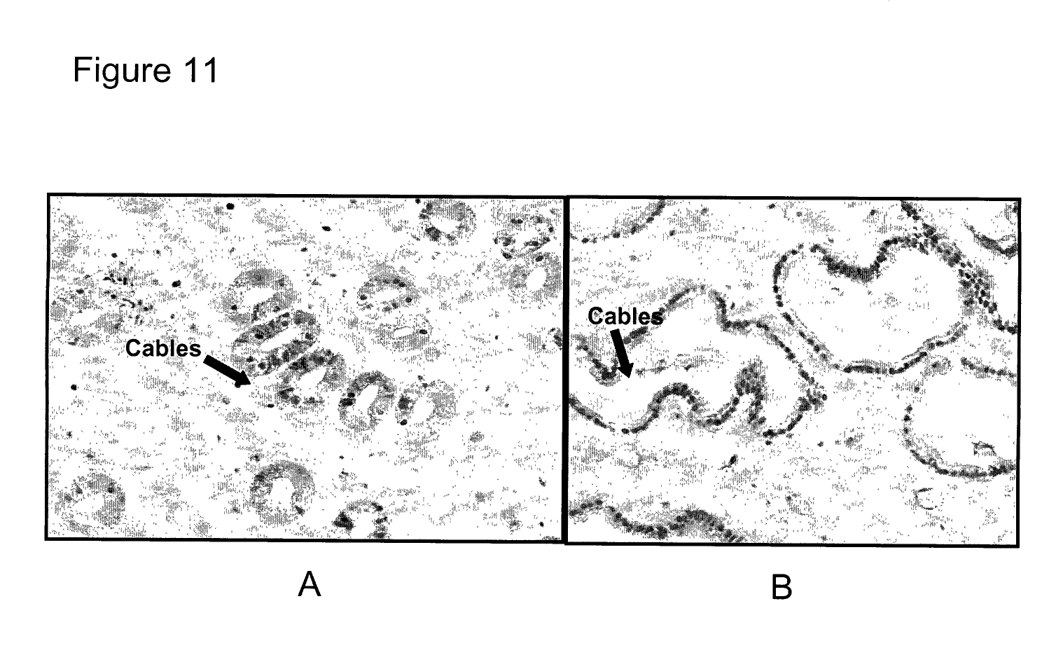

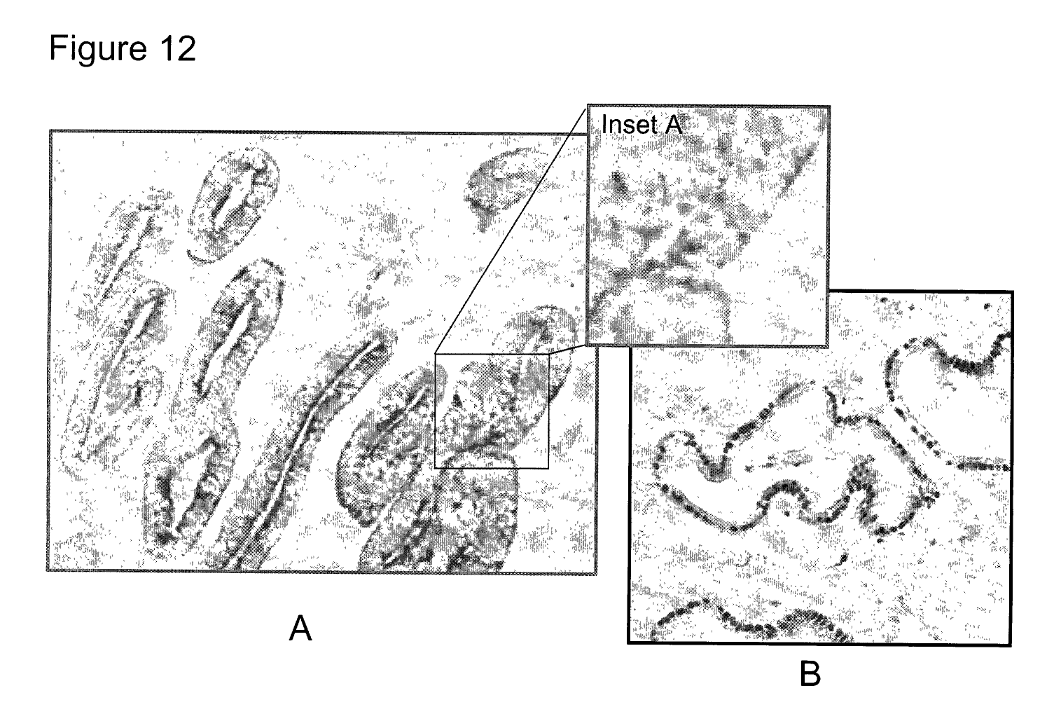

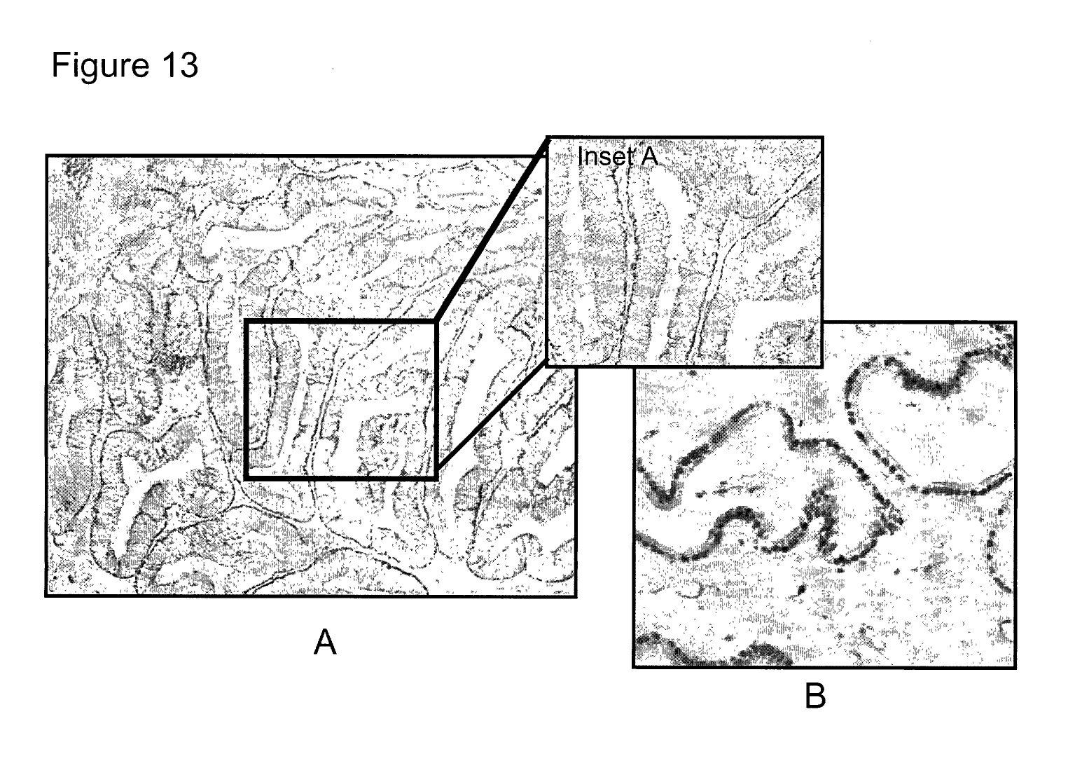

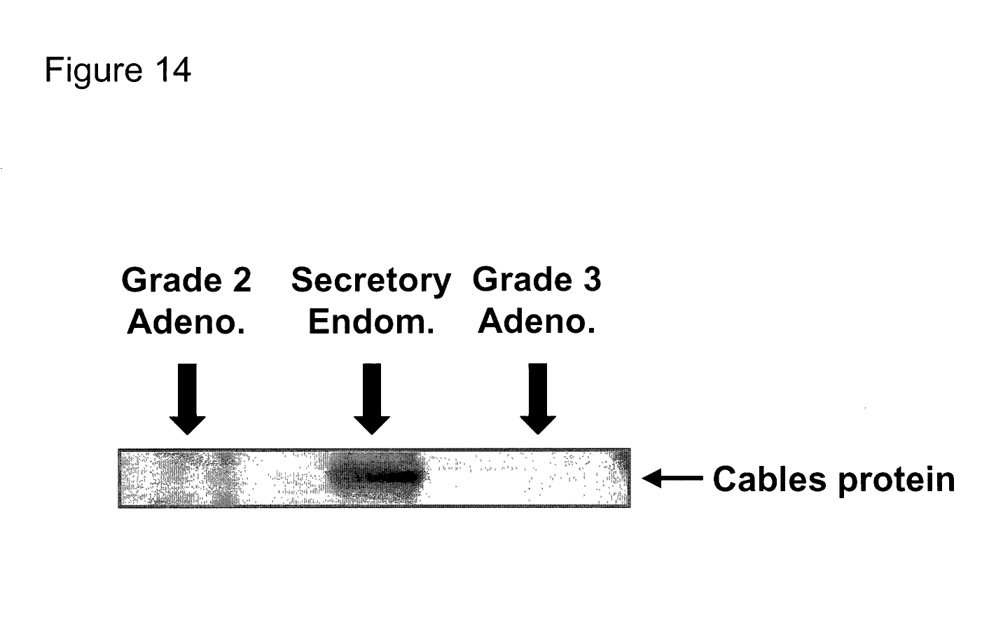

Cables expression was also evaluated in endometrial tissue samples from normal, hyperplastic, and malignant human endometrium. Thirty paraffin embedded specimens from patients were examined by immunohistochemistry (IHC). Cables expression was seen in all normal endometrial samples (n=13; FIG. 11), whereas Cables expression was lost in greater than 90% of the endometrial hyperplasia and cancer specimens (FIG. 12). None of the endometrial carcinomas expressed Cables protein (n=12; see FIGS. 12, 13, and 14). Western analysis confirmed that the Cables protein could be identified in normal, but not malignant endometrial tissue (FIG. 14). Northern analysis of these same tissue specimens identified Cables mRNA transcripts in normal, but not malignant endometrium. Moreover, tumors generated in nude mice from HES cells did not express Cables protein when evaluated by IHC.

Hormonal regulation of Cables expression has also been investigated in endometrioid tissue. During the menstrual cycle, follicular stimulating hormone stimulates the ovary to produce increasing amounts of estrogen. In turn, estrogen causes endometrial tissue to build up (or proliferate), lining the interior of the uterus. During this phase, Cables expression is down-regulated (FIG. 11A). As the cycle progresses into the secretory phase, however, progesterone becomes the dominant hormone and acts to up-regulate Cables expression, thereby inhibiting cellular proliferation (FIG. 11B). Progesterone inhibits cellular proliferation by promoting terminal differentiation of the cells of the endometrium, thereby augmenting degeneration of the endometrium and sloughing of the cells during menses.

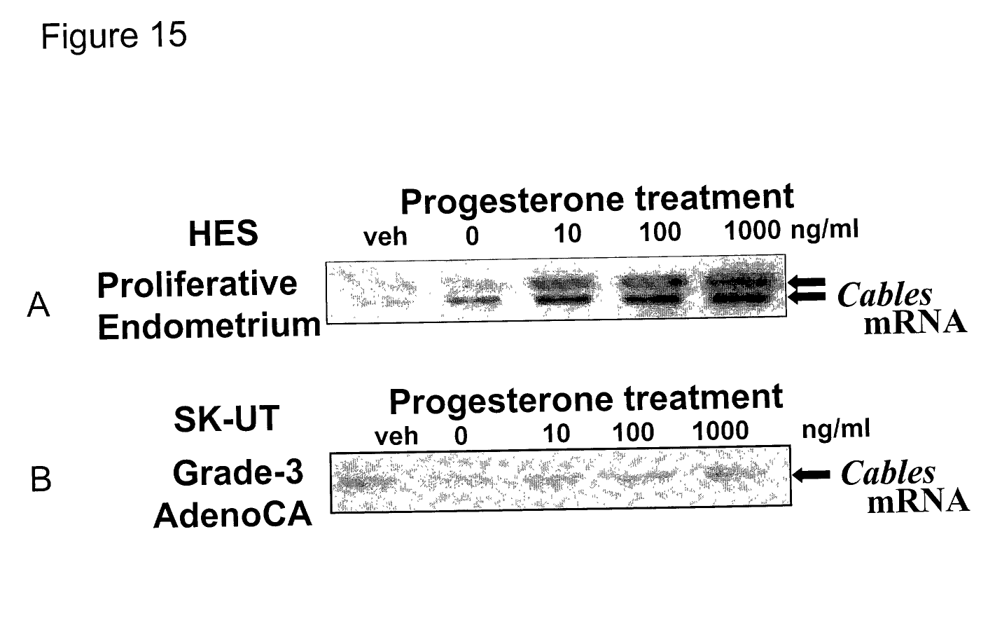

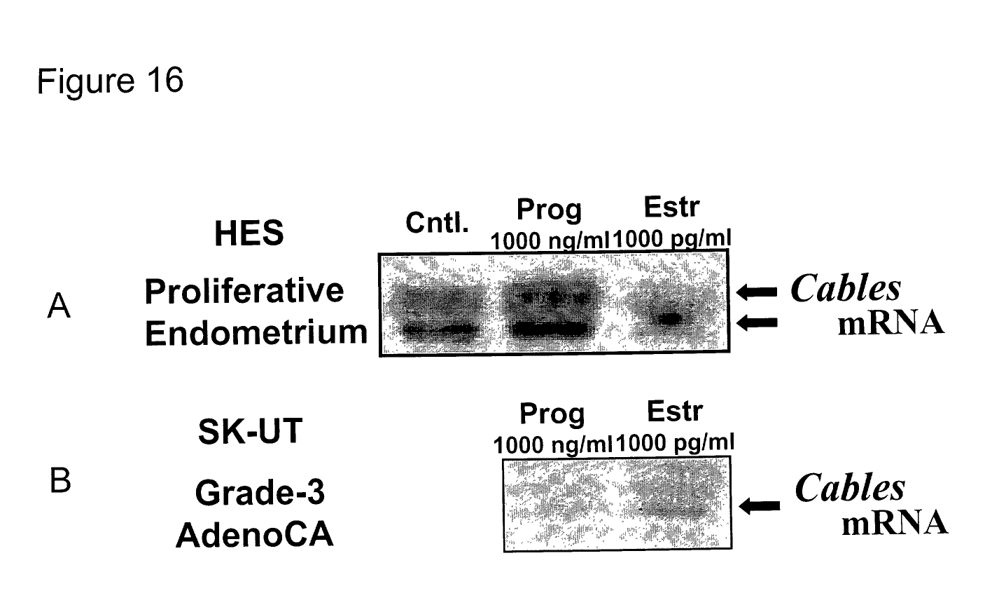

Cell lines have been used to investigate the effect of hormonal regulation on Cables expression, as well as the effect of Cables overexpression on these cells. We have isolated RNA and protein from HES, Ishikawa, and SK-UT2 endometrial cells lines for analysis. The influence of progesterone and estrogen on Cables expression was evaluated in these cells. Progestin treatment caused a dose dependent elevation in Cables mRNA levels in HES cells, a benign proliferative human endometrium cell line, and Ishikawa cells, which are derived from a G1 endometrioid tumor and are well differentiated, as compared to untreated controls (FIG. 15A). Western analysis confirmed that a protein product was present. Conversely, estrogen reduced the levels of Cables mRNA expression in these cell lines (FIG. 16A). There is was no hormonal effect on Cables mRNA expression in SK-UT2 cells, which are derived from a G3 human uterine cancer (FIGS. 15B and 16B). SK-UT2 cells appear to have lost one Cables transcript, while the second transcript was expressed at much lower levels than that which was observed in HES or Ishikawa cells.

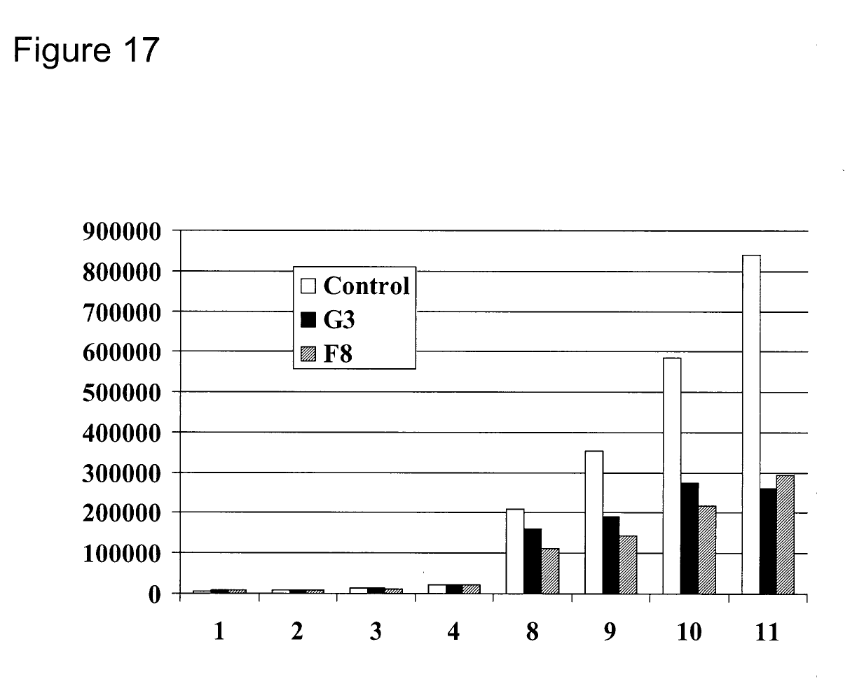

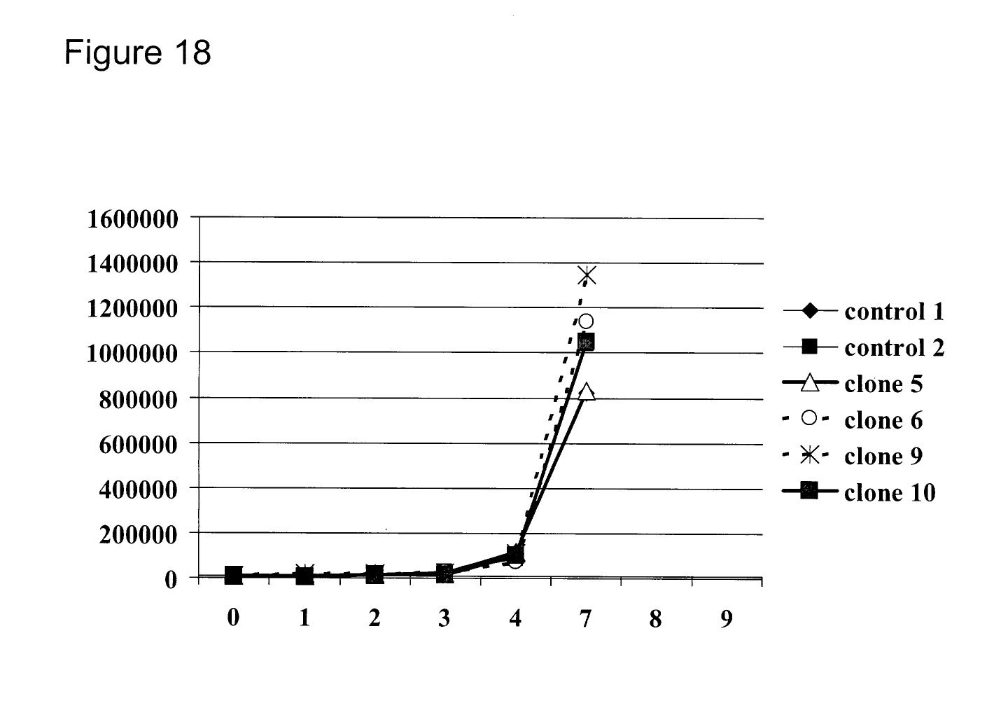

We also examined the overexpression of Cables in the HES and SK-UT2 cells lines. Overexpression of Cables in HES cells resulted in a marked reduction in the level of cellular proliferation (FIG. 17). In contrast, the proliferation rate of the SK-UT2 cells was not different from the respective parent cell line (FIG. 18). The transfected cell lines were stable and the results were repeatable. As the suppression of cellular proliferation by Cables is correlated with endometrioid-derived tumors, it is likely that the SK-UT2 cells are not of an endometrioid origin.

To further understand the genetic basis of Cables function, we examined LOH, as is described above, in endometrioid cancers. Unlike head, neck, and colon tumors, endometrioid cancers do not exhibit LOH specifically for Cables. After evaluating 10 endometrial cancers and surrounding normal tissue, there was no evidence to suggest that this tumor was related to LOH. Examination of the Cables gene in endometrial tumors has revealed the presence of mutations in the Cables gene. These mutations range from a single base pair deletion to large deletions (>300 bp). These results indicate that Cables is a regulator of endometrial hyperplasia and cancer.

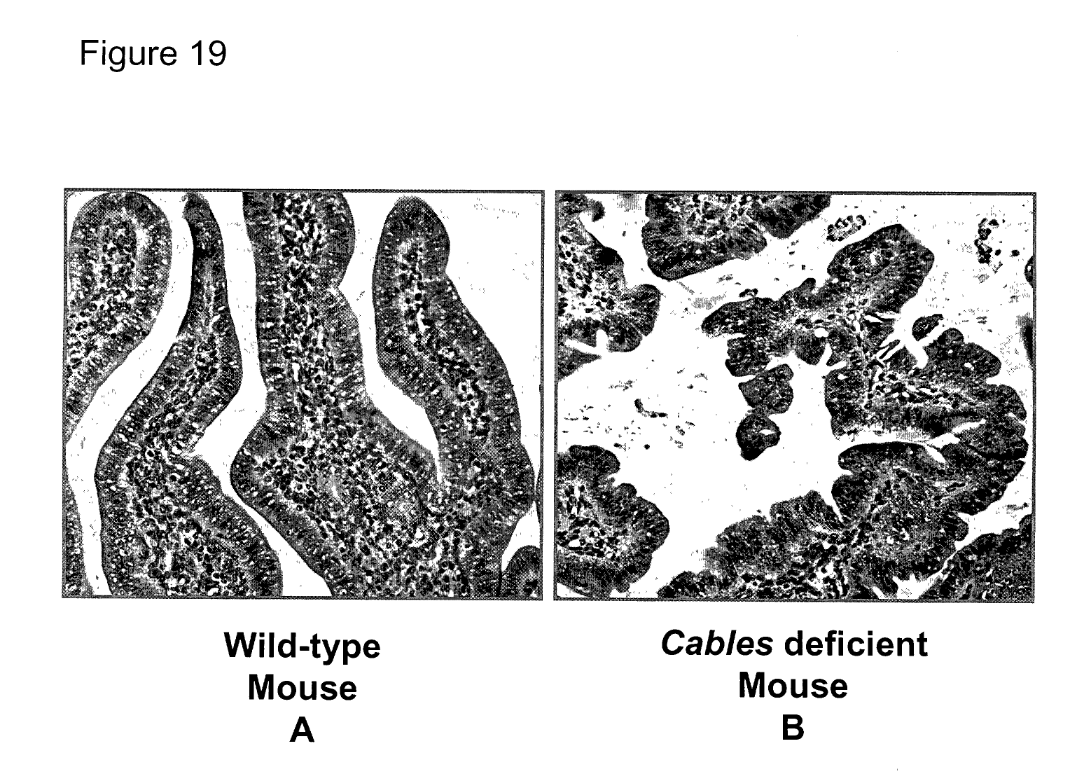

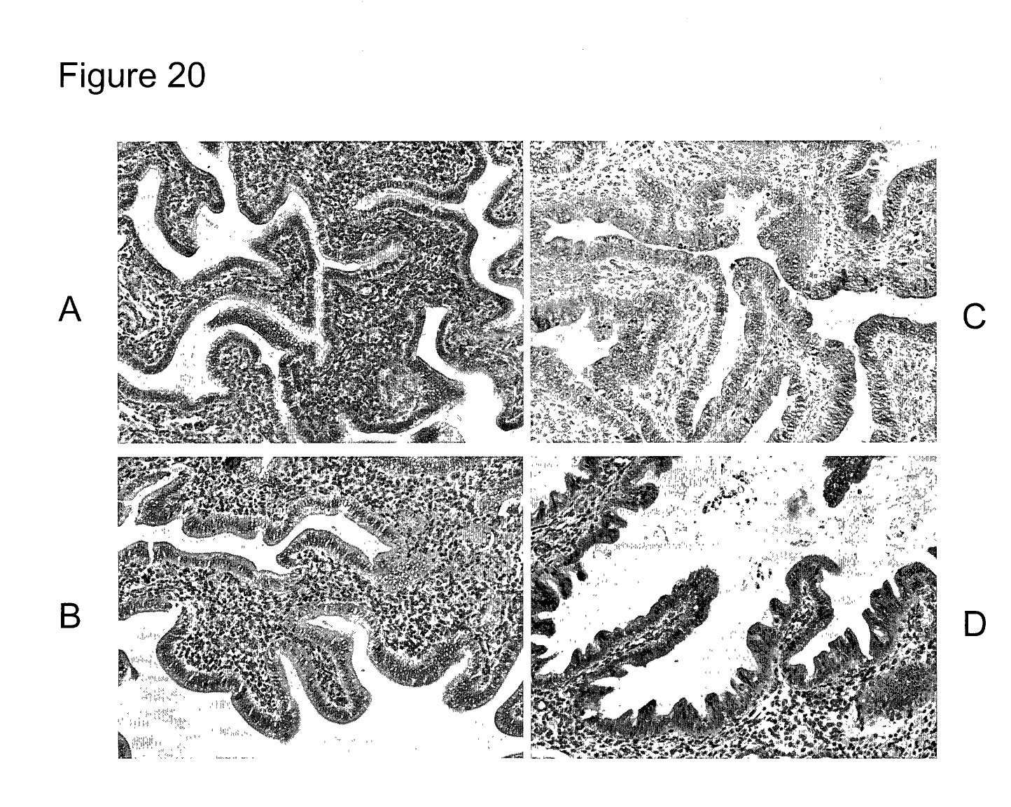

We have also generated a mutant mouse deficient in Cables using standard techniques. Female mice from this strain were challenged with gonadotropins to stimulate follicular development, the consequence of which would be elevated estrogen, which has been implicated as a progenitor of endometrial cancer. Within days, the endometrium of these mice demonstrated atypical hyperplasia, often considered the precursor to endometrial cancer (compare FIGS. 19A, 20A, and 20B to FIGS. 19B, 20C, and 20D). Within three months, these mice developed early carcinoma of the endometrium. These results further support the hypothesis that Cables is involved in control of cell growth, and loss of Cables causes uncontrolled cell growth and promotes tumorigenesis.

Loss of Cables Expression Is Associated with Ovarian Cancer

In addition to the results provided above in connection with head and neck squamous cell cancer and endometrial cancer, we have also determined that greater than 70% of ovarian serous tumors exhibit loss of Cables nuclear expression. Studies of ovarian cancers has demonstrated that LOH occurs at numerous chromosomal locations including 3p, 5q, 6, 7p, 8, 9q, 11p, 13q, 17, 18q, 21q, and 22q; however, loss or deletion of chromosome 18q, the location of the Cables gene, is one of the common chromosomal abnormalities in ovarian cancer and has been reported to occur in 25% to 40% of ovarian carcinomas.

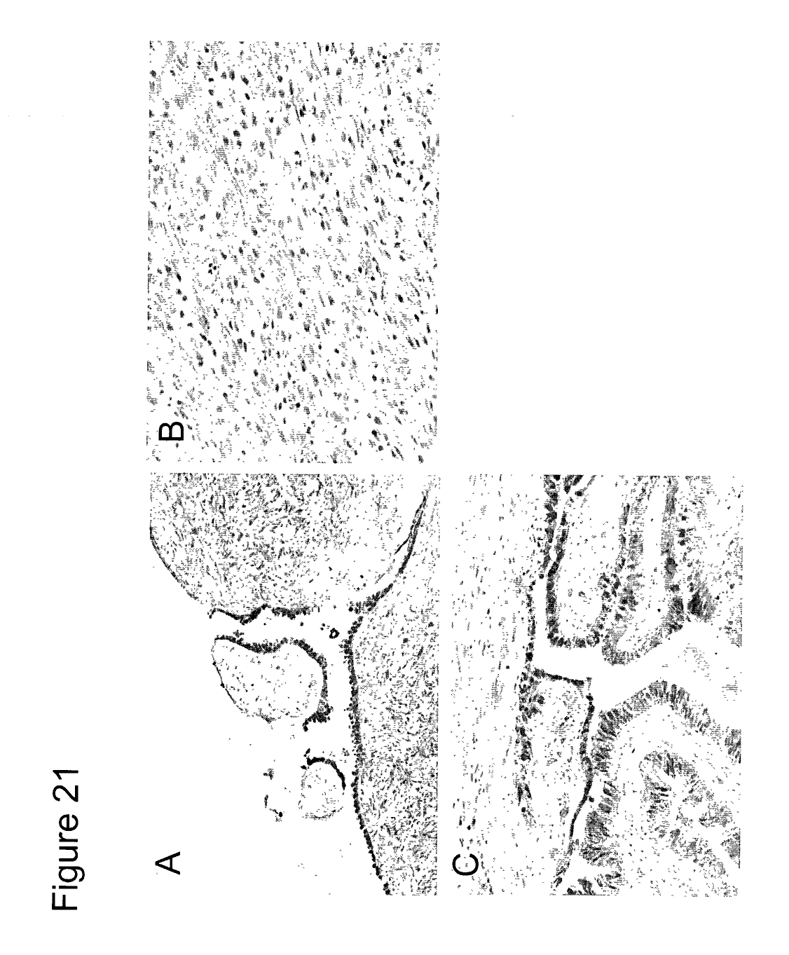

To determine the role of Cables in ovarian cancer, Cables expression was examined in the four most common subtypes of ovarian carcinomas: serous, endometrioid, mucinous, and clear cell. We also examined mucinous and serous borderline tumors. We first examined normal ovarian surface epithelium (FIG. 21A), ovarian stromal cells (FIG. 21B), and fallopian tube epithelium (FIG. 21C), which showed strong nuclear staining for Cables using the affinity purified anti-Cables antisera. In addition, inflammatory cells including lymphocytes and plasma cells, if present, were positive for Cables staining as well. No staining was detected with negative controls or the preimmune sera.

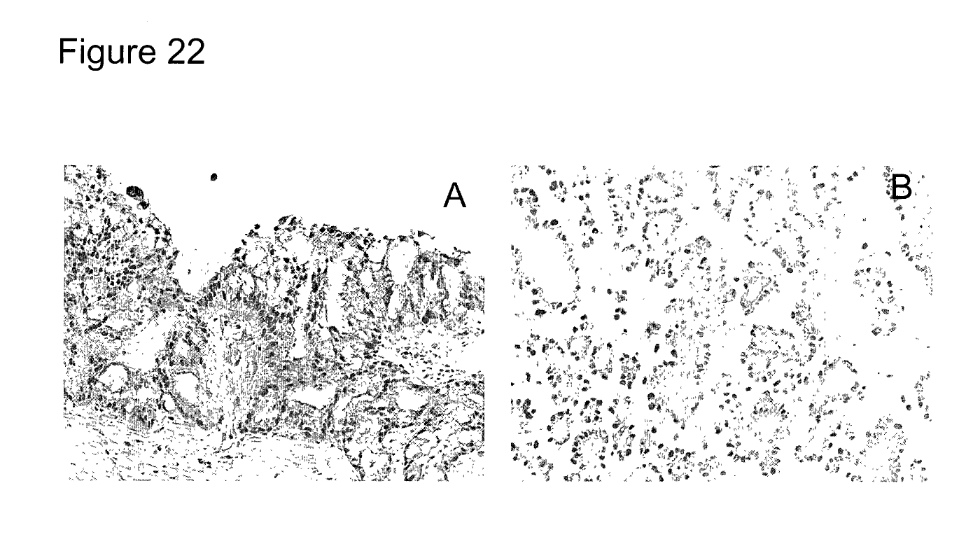

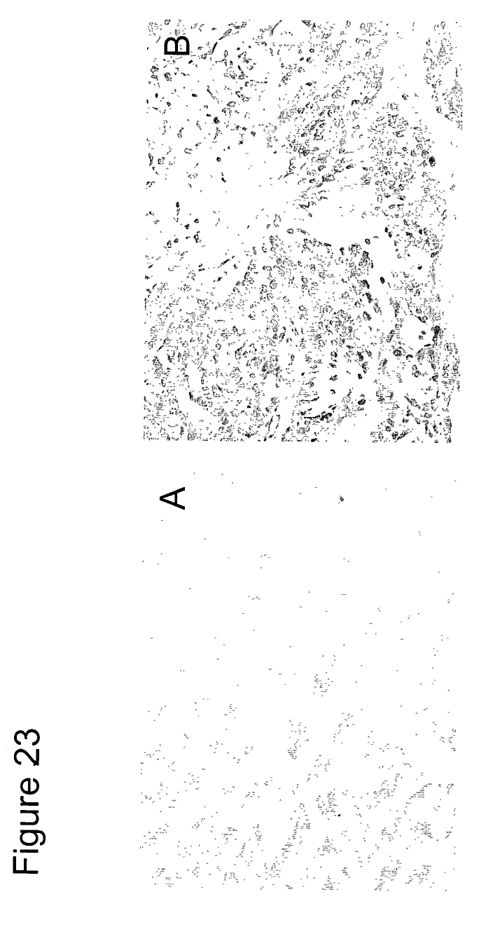

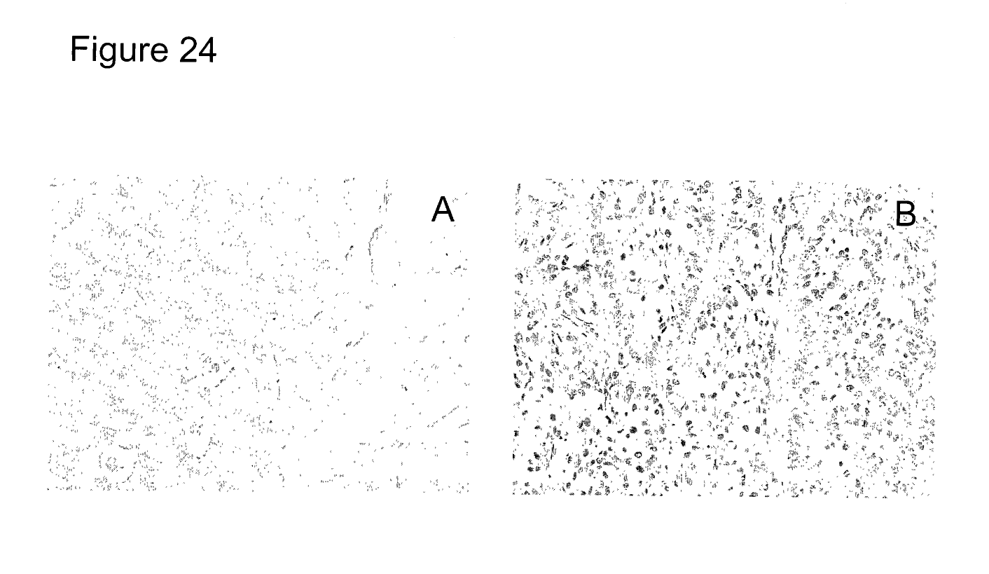

We observed strong Cables nuclear staining in all mucinous borderline tumors and carcinomas (FIG. 22A) and clear cell carcinomas (FIG. 22B). In contrast, 11 out of 14 serous carcinomas (79%) showed complete (9 cases; FIG. 23A) or partial (2 cases) loss of Cables staining. The remaining 3 cases showed positive Cables staining (FIG. 23B). In the 14 borderline serous tumors, 11 cases showed positive Cables staining, and the remaining 3 cases showed focal partial loss of Cables. Five out of 10 endometrioid carcinomas (50%) showed complete (3 cases; FIG. 24A) or partial (2 cases) loss of Cables staining, and the rest showed positive Cables expression (FIG. 24B). The benign surface epithelium and background fibroblasts and inflammatory cells adjacent to tumor cells, if present, showed positive nuclear staining.

Immunohistochemical stains for p53 and MIB1 (prognostic markers for the detection of cancer) were also performed in all 14 serous carcinomas. Strong positive p53 staining, as well as a relatively high percentage of MIB1 positive cells (20-80%), was detected in all cases. There was no statistically significant difference in p53 and MIB1 staining between Cables-positive and negative cases.

Clinical and Pathological Findings

Serous and endometrioid ovarian carcinomas showed variable expression of Cables; therefore, we tried to further evaluate any possible correlation among Cables positive and negative tumors and clinicopathologic features associated with these tumors (see Table 3 and 4). No correlation was noted between loss of Cables staining and histologic grade, clinical stage, or outcome among the serous or endometrioid carcinomas.

TABLE 3
Correlation of Cables Expression with Clinicopathologic Findings
of Ovarian Endometrioid Adenocarcinoma

| Age | Histologic | | | Cables |
|---|---|---|---|---|
| (years) | Grade | Clinical Stage | Follow-Up | Staining |
| 81 | 2/3 | T1aN0M0 | AW | −/+ |
| 68 | 2/3 | T1cN0M0 | AW | + |
| 60 | 2/3 | T1cN0M0 | AW | − |
| 82 | 3/3 | T3bN0M0 | DOD | −/+ |
| 41 | 3/3 | T1bN1M0 | DOD | + |
| 57 | 1/3 | T1bN0M0 | AW | + |
| 41 | 2/3 | T1bN0M0 | AW | + |
| 80 | 3/3 | T1cN0M0 | AW | − |
| 69 | 2/3 | T1cN0M0 | DOD | − |
| 54 | 2/3 | T1aN0M0 | AW | − |

AW: alive well; DOD: died of disease; −/+: partial loss of staining

TABLE 4
Correlation of Cables Expression with Clinicopathologic Findings
of Ovarian Serous Carcinoma

| Age | Histologic | | | Cables |
|---|---|---|---|---|
| (years) | Grade | Clinical Stage | Follow-Up | Staining |
| 62 | 3/3 | T3cN0M0 | DOD | −/+ |
| 71 | 3/3 | T3cN1M0 | DOD | − |
| 42 | 3/3 | T3cN0M0 | AWD | − |
| 32 | 3/3 | T3cN1M1 | DOD | − |
| 78 | 3/3 | T3cN1M0 | DOD | − |
| 74 | 3/3 | T3cN1M0 | DOD | + |
| 59 | 3/3 | T3cN0M0 | DOD | − |
| 49 | 3/3 | T3cN0M1 | DOD | − |
| 36 | 3/3 | T3cN1M0 | DOD | − |
| 48 | 3/3 | T3cN0M0 | DOD | + |
| 74 | 3/3 | T3bN1M0 | AWD | − |
| 51 | 3/3 | T3cN0M1 | DOD | − |
| 60 | 3/3 | T3N0M1 | DOD | + |
| 40 | 3/3 | T3cN1M1 | DOD | −/+ |

AWD: alive with disease; DOD: died of disease; −/+: partial loss of staining

Cables cDNA Expression in Ovarian Cancer

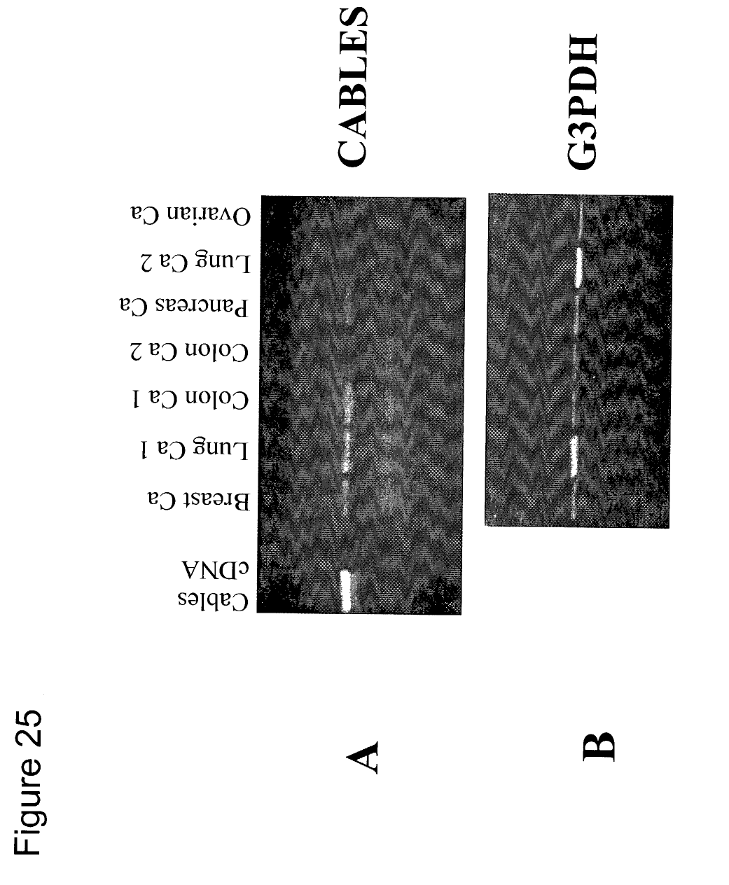

As is described above, we again isolated first strand cDNA from 7 human tumor xenografts (Clontech), including lung, colon, breast, pancreas and ovary tumors and screened them with Cables-specific primers. Again, G3PDH primers were used as a control. Four tumors showed approximately equal levels of Cables cDNA while three tumors (colon, lung, and ovary) showed significantly decreased Cables cDNA (FIG. 25A). G3PDH cDNA levels were approximately the same in all of the tumors (FIG. 25B). These results support the correlation between loss of Cables mRNA and ovarian cancer.

The primary function of Cables appears to be regulating cell growth. The loss of chromosome 18q, which includes the genetic locus of Cables, in ovarian cancers strongly supports the role of Cables as a candidate tumor suppressor gene. The role of Cables in ovarian cancer is also supported by our studies with colorectal adenocarcinoma, head and neck squamous cell carcinoma, and endometrial carcinoma (described above).

The loss of Cables expression was found in a high percentage of ovarian serous and endometrioid carcinomas, and was observed as not only a loss of Cables at the polypeptide level, but also at the mRNA level, as determined by RT-PCR analysis in a human ovary tumor xenograft. In contrast, all clear cell carcinomas, mucinous borderline tumors and carcinomas, and the majority of serous borderline tumors showed strong Cables nuclear staining, indicating that while Cables is involved in the development of ovarian serous and endometrioid carcinoma, the tumorigenesis of clear cell and mucinous tumors is likely mediated through mechanisms that are different from serous and endometrioid carcinomas.

Loss of Cables expression has been also observed in numbers of cancers in other locations, including colorectal adenocarcinoma, head and neck squamous cell carcinoma, lung cancer, esophageal cancer, stomach cancer, endometrial cancer, and bladder transitional cell carcinoma. In addition, our RT-PCR analysis also showed loss of Cables mRNA in colon and lung cancers.

Without being bound to a particular theory, there are a number of reasons why loss of Cables in ovarian cancer could lead to uncontrolled cell proliferation. Cables interacts with many proteins involved in growth control including cdk2 and p53. Cables enhances a cdk2 inhibitory phosphorylation by the Wee1 protein kinase, which decreases cdk2 activity. Therefore loss of Cables may disregulate cdk2 function, and enhance tumor formation. In addition, it is well characterized that almost all tumors develop resistance to growth inhibitory signals and genes involved in this resistance almost always relate to the retinoblastoma gene product, pRB. Mutations in pRB, viral infections, alternation of genes that normally regulate pRB are common mechanisms of pRB inactivation. Since pRB is regulated by cdks, loss of Cables, which leads to disregulated cdk2 activity, would also cause pRB inactivation.

P53 mutation is common in ovarian serous carcinoma. Published studies have also shown that Cables interacts with p53, and may enhance p53 induced apoptosis. To test whether there was a correlation between p53 mutations and Cables loss, we studied p53 expression in the serous carcinomas. Our results demonstrated that all serous carcinomas in our series were positive for p53, regardless of Cables expression. This indicates that loss of Cables is a late event in tumorigenesis. No significant difference in MlB1-positive cells are detected between Cables positive and negative serous carcinomas in our series. No correlation of Cables expression versus tumor grade, clinical stage, or prognosis was seen.

Loss of Cables Expression Is Associated with Prostate Cancer

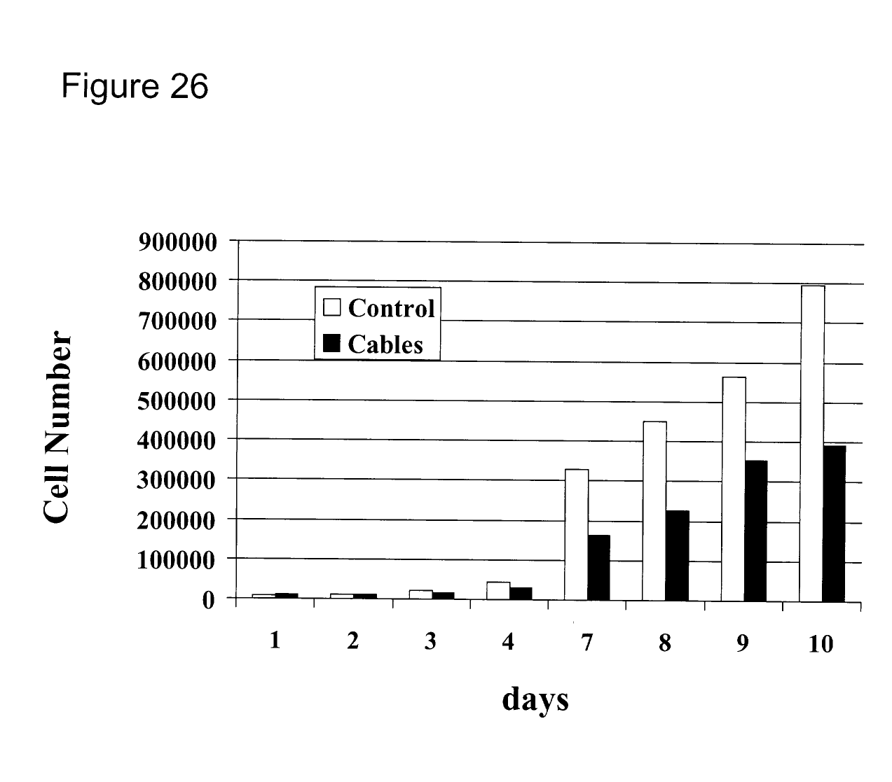

In addition to the above results, we also investigated the effect of Cables in prostate (Du-145) and colon (CaCo) derived cell lines. Following stable transfection, both the Du-145 and the CaCo cells demonstrated a marked inhibition (>50%) of cell proliferation rates compared to untransfected or mock transfected controls (FIG. 26).

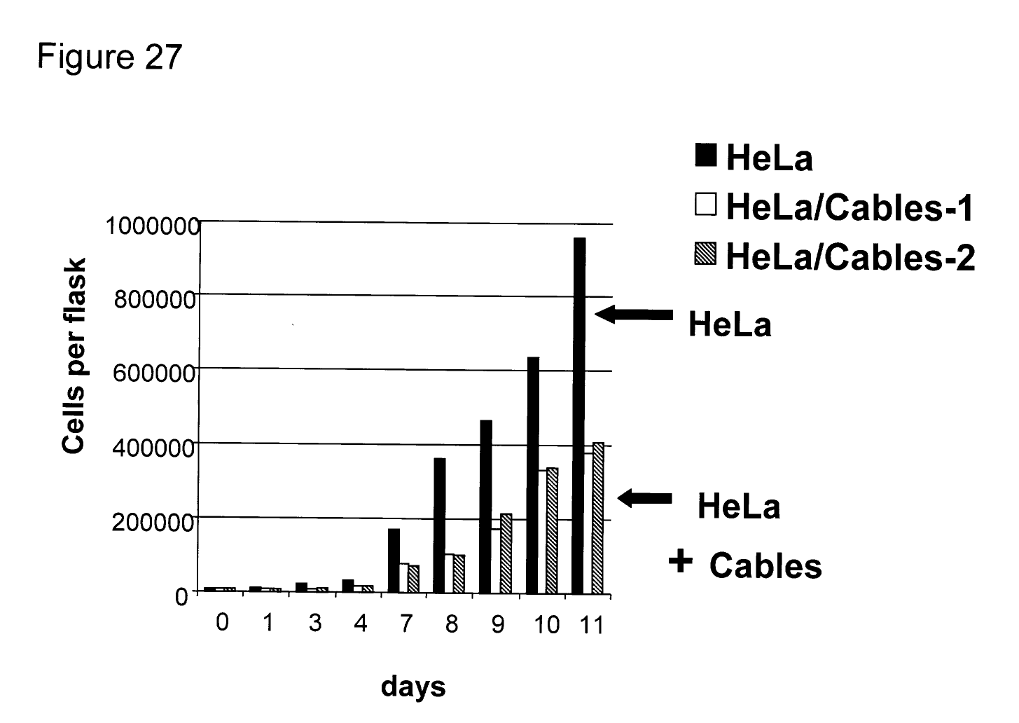

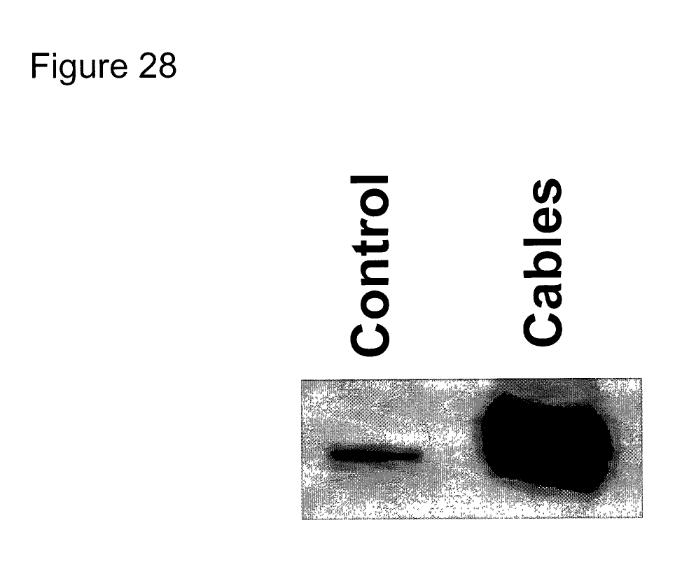

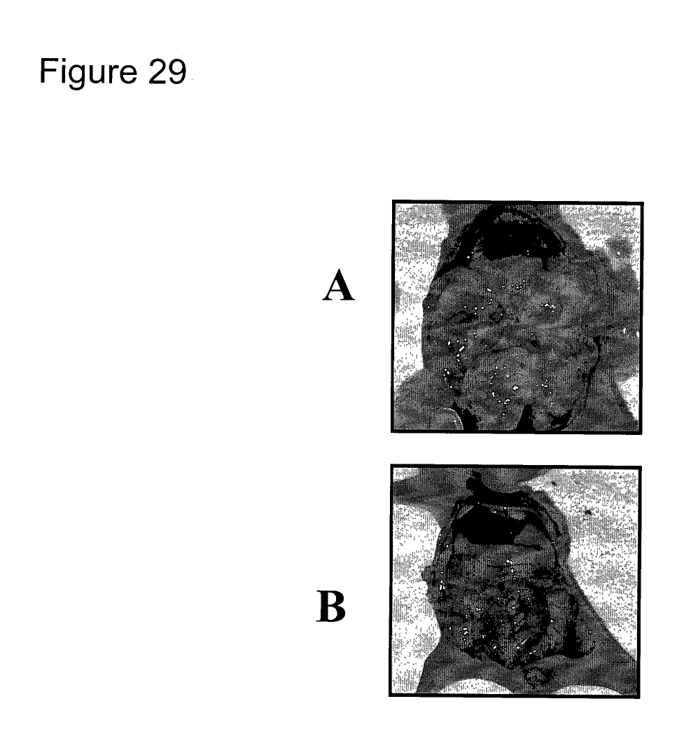

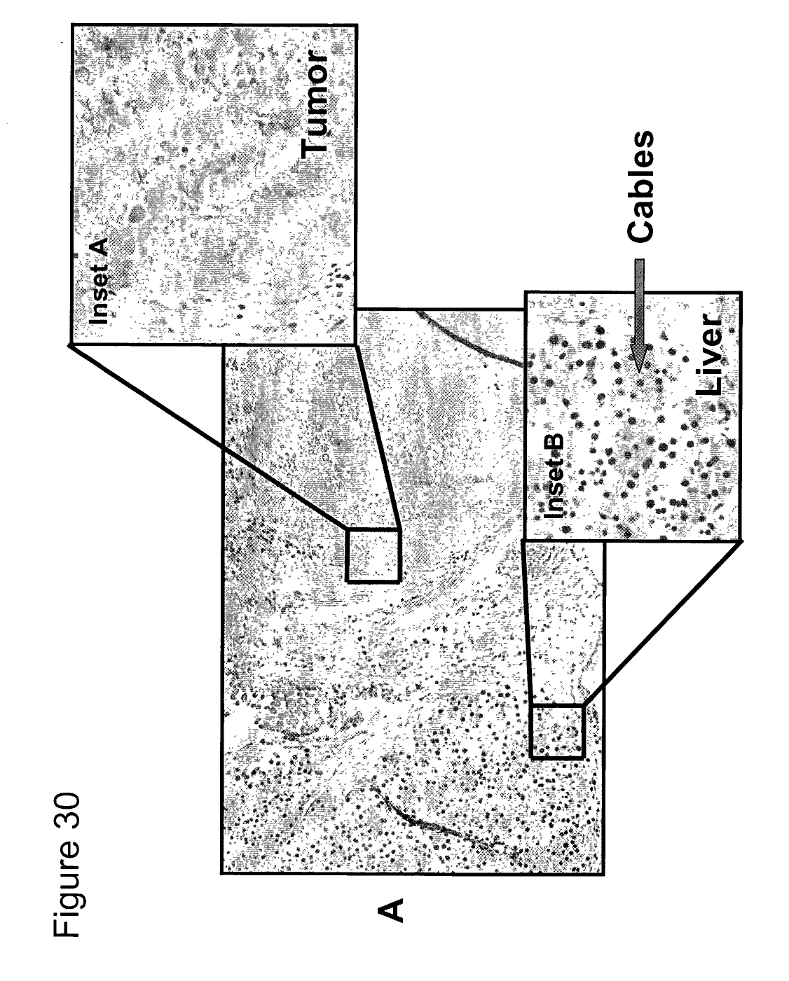

We also investigated whether Cables overexpression in HeLa cells would inhibit cellular proliferation. As seen with Du-145 and CaCo cells, HeLa cells also demonstrated a marked inhibition (>50%) of cell proliferation rates following stable transfection, as compared to untransfected or mock transfected controls (FIG. 27). Overexpression of Cables was demonstrated by western blot (FIG. 28). To verify that Cables overexpression suppresses tumor growth in vivo, HeLa cells stably-expressing Cables were transplanted into nude mice. Seven out of nine mice administered HeLa cells with low expression of Cables developed tumors (FIG. 29A), while only 2 out of 9 mice administered HeLa with high expression of Cables developed tumors (FIG. 29B). Examination of the tumor tissue revealed that Cables expression was nearly absent. (FIG. 30). These results confirm the inhibitory proliferative effect of Cables expression in tumor cells.

Materials and Methods

The above experiments were carried out using the following materials and methods.

Cell Culture and Generation of Stable Cell Lines

HeLa and COS7 cells were propagated in Dulbecco's modified Eagle's medium with 4.5 g/l of glucose, 10% fetal calf serum, and penicillin/streptomycin. Transient transfections in COS7 cells were performed using the calcium phosphate method with 10-20 μg of total DNA. For generation of stable cell lines, full length Cables was cloned into the pCIN4 vector and the resulting plasmid transfected into HeLa cells using lipofectamine (Life Technologies, Inc.). Stable cell lines were selected by growth in 50 μg/ml G418. Control cells were made by transfection of vector alone. The growth rates of both the Cables and control cells were determined by seeding 1×10⁴ cells/well and counting the cells in triplicate for up to 13 days. Colony formation efficiency was determined by plating single cells at 1×10³/100 mm dish and incubating plates undisturbed for 10 days to allow for colony formation. Cells were fixed, Giemsa stained, and colonies of 25 or more cells counted as positive. Seeding efficiency was determined by seeding 2×10⁶ cells/100 mm dish and counting the number of adherent cells after 6 hours. To examine anchorage independent growth, single cells (1×10³ cells/60 mm dish) were dispersed in 0.36% Nobel Agar in DMEM-20% fetal bovine serum (FBS) and layered onto a 0.6% agar base. Media was added every three days and colonies counted after 5 weeks.

DNA Constructs

Full length Cables was obtained from a mouse neonatal brain cDNA library and a human fetal brain cDNA library (Clontech) and sequenced. For production of bacterial fusion proteins, the Cables cDNA was subcloned in-frame into pGEX-4T-2 (Pharmacia) at the BamH1 site. The cdk2, Wee1, and cdk2Y15F constructs were a gift of L. H. Tsai (Harvard Medical School, Boston, Mass.).

Protein Analysis

Cell lysate was produced in E1A lysis buffer (50 mM Tris-HCL [pH 7.5], 250 mM NaCl, 5 mM EDTA [pH 8.0], 0.1% Nonidet P-40, 5 mM DTT, 10 mM NaF, 1 mM PMSF, 1 μg/ml aprotinin, 1 μg/ml leupeptin, and 1 μg/ml Na₃ VO₄). Proteins were analyzed by direct western blotting (50 μg/lane) or blotting after immunoprecipitation. Cell extracts were immunoprecipitated with anti-cdk2 (pAb M2; Santa Cruz Biotechnology) and anti-Cables (pAb 64; Zukerberg et al., Neuron 26:633-646 (2000)). Immunoprecipitates were collected by binding to protein A-sepharose. Western blots were probed with anti-cdk2 (mAb D12; Santa Cruz Biotechnology), anti-cdc2 (pAb H-297; Santa Cruz Biotechnology), anti-Cables (pAb 64), anti-phosphotyrosine (mAb 4G10; Upstate Biotechnology), anti-cyclin E (mAb HE12; gift of E. Harlow), anti-cyclin A (mAb C160; gift of E. Harlow), anti-p21 (mAb CP74; gift of E. Harlow), anti-p27 (mAb HBB6; gift of E. Harlow) and anti-phospho-Cdc2 (Tyr15; pAb, New England Biolabs).

Cell Fractionation

Cells were resuspended in STM buffer (10 mM Tris-HCL [pH 8.0], 25 M sucrose, 10 mM MgCl₂, 0.1 mM DTT, 10 mM NaF, 1 mM PMSF, 1 μg/ml aprotinin, 1 μg/ml leupeptin, and 1 μg/ml Na₃ VO₄) and homogenized. Lysates were centrifuged at 600×g for 15 min. Supernatent was centrifuged at 100,000×g, and the recentrifuged supernatent with 0.05% NP-40 was saved as the cytoplasmic fraction. The pellet from the initial centrifugation was resuspended in STM buffer plus 0.5% NP40 and centrifuged at 600×g for 15 min. The supernatent was saved as the membrane fraction and the pellet was saved as the nuclear fraction, which is resuspended in radioimmunoprecipitation assay (RIPA) buffer.

In Vitro Kinase Assay

Kinase assays were performed by washing immunoprecipitates three times with lysis buffer and once with kinase buffer (50 mM HEPES [pH 7.0], 10 mM MgCl₂, and 1 mM DTT). Cdk2 levels were equalized by western blotting before the kinase assay. Subsequently, the beads were incubated with kinase buffer containing 0.5 μg of histone H1 and 5 μCi of [γ-³²P] ATP in a final volume of 50 μl at room temperature for 30 min.

Cloning of Human Cables cDNA and FISH Mapping of Cables

The human Cables cDNA was isolated from a human fetal brain cDNA library using the mouse Cables cDNA and was used as a probe for chromosomal in situ hybridization. Chromosomal slides were prepared from lymphocytes isolated from human blood and cultured in minimal essential media supplemented with 10% FCS and phytohemagglutinin. The lymphocyte cultures were treated with bromodeoxyuridine (0.18 mg/ml) to synchronize the cell population. Synchronized cells were released from the block and recultured for 6 h. Slides were made using hypotonic treatment, fixation and air-drying. The human Cables cDNA was biotinylated with dATP using the Life Technologies, Inc. BioNick labeling kit and used for fluorescent in situ hybridization (FISH). The procedure for FISH detection was performed according to standard procedures (Heng et al. Proc. Natl. Acad. Sci. USA 89:9509-9513, 1992; Heng and Tsui, Chromosoma 102:325-332, 1993). FISH signals and DAPI banding pattern was recorded separately by photographs and the assignment of the FISH mapping data with chromosomal bands was achieved by superimposing FISH signals with DAPI banded chromosomes.

Reverse Transcription (RT)-PCR of Cables in Human Tumors

The Cables mRNA of human tumor xenografts was studied using a panel of eight normalized, first-strand cDNA preparations from human tumor xenografts (Human Tumor MTC Panel; Clontech) according to directions. Cables PCR primers were 5′-GCAGGAGGACTGTGGCCTTGAGGAG-3′ (SEQ ID NO: 3); 5′-CTGTGTGCTGGGGCATGTGTGCTGT-3′ (SEQ ID NO: 4); and 3′-GGCCCTTGGCTGTCCTCGGGGCCAGTG-5′ (SEQ ID NO: 5); which amplified the very C-terminal 350 and 250 bp of the open reading frame. G3PDH PCR primers (included in kit) were used as a cDNA normalization control.

Immunohistochemistry (IHC)

GST fusion proteins were expressed in Escherichia coli and purified with GSH beads. A specific antibody was raised against a GST-tagged human Cables in rabbits and affinity purified. The antibody recognized a protein of about Mᵣ 70,000 in cell lysates on SDS-PAGE that comigrated with Cables synthesized in rabbit reticulocyte lysates in vitro and that was recognized by the previously described anti-mouse Cables antisera (Zuckerberg et al., Neuron 26:633-646 (2000). Formalin fixed, paraffin-embedded sections of normal and tumor tissue were stained with affinity purified anti-Cables antisera, at a 1:200 dilution, using a microwave enhanced the avidin-biotin staining method (Hsu et al., J. Histochem. Cytochem. 29:577-580, 1981;Yang et al., Am. J. Pathol., 145:86-96, 1994). Negative control sections were immunostained under the same conditions substituting preabsorbed antisera and preimmune rabbit antisera for primary antibodies. The specificity of the affinity purified antisera was demonstrated by lack of staining with preabsorbed antisera and preimmune antisera, and strong staining of COS7 cells transfected with Cables with little to no staining of nontransfected cells, which contained little Cables by western blot.

The nuclear expression of Cables in endometrioid tissue was assessed by immunohistochemical staining on formalin-fixed, paraffin-embedded tissue sections of 28 serous tumors (14 carcinomas, and 14 borderline tumors), 10 mucinous tumors (5 carcinomas and 5 borderline tumors), 10 endometrioid carcinomas, and 10 clear cell carcinomas. Complete loss of Cables was defined as negative nuclear staining of all the tumor cells with positive staining of background normal tissue including fibroblasts and inflammatory cells. Partial loss was defined as both negative and unequivocal positive stained tumor cells in the same tissue section despite even staining of background normal tissue. All cases were evaluated by three pathologists.

In addition, immunohistochemical stains for p53 and MIB1 were performed in the 14 serous carcinomas using the avidin-biotin peroxidase technique. Paraffin-embedded sections were deparaffinised and heated in a pressure cooker at 120° C. for 5 minutes for antigen retrieval. The sections were subsequently incubated for 5 minutes in 3% hydrogen peroxide to quench endogenous peroxidase activity. The sections were then incubated with the primary antibodies (1:15 dilution for anti-p53 and 1:40 for anti-MIB1), followed by secondary antibodies and avidin-biotin staining. The intensity of p53 staining was graded as negative, weakly positive, positive, and strongly positive.

cDNA Expression

The Cables mRNA of human tumor xenografts was studied using a panel of 7 normalized, first strand cDNA preparations from human tumor xenografts (Human Tumor MTC Panel; Clontech) according to directions. Cables PCR primers were 5′-GCAGGAGGACTGTGGCCTTGAGGAG-3′(SEQ ID NO: 6), 5′-CTGTGTGCTGGGGCATGTGTG-CTGT-3′(SEQ ID NO: 7), and 3′-GGCCCTTGGCTGTCCTCGGGGCCAGTG-5′(SEQ ID NO: 8), which amplified the very COOH-terminal 350-bp and 250-bp of the open reading frame. G3PDH PCR primers (included in the kit) were used as a cDNA normalization control.

Loss of Heterozygosity (LOH)

To examine loss of heterozygosity, archival DNA was extracted from formalin-fixed specimens by using standard methods (Young et al., Oncogene 8:671-675, 1993). Briefly, sections 4 μm thick were mounted on slides and non-neoplastic tissue removed with a clean blade by superimposing the unstained section with the corresponding stained section. The tissue was deparaffinized and rehydrated through xylene and alcohol, scraped into a microfuge tube, and DNA extracted with phenol and chloroform mixture. DNA was also extracted from paired normal tissue in all cases. Loss of heterozygosity in the region of the Cables gene on chromosome 18q was examined by using the highly polymorphic markers D18S44 and D 18s1107 which map to 18q11. Loss was scored as previously described (Young et al., Oncogene 8:671-675, 1993).

Examination of Endometrial Tissues and Cell Lines for Cables Expression

Paraffin embedded sections of endometrium from 30 patients were examined using IHC. In a subset of cases, RNA was isolated and analyzed by northern blot to determine if the absence of Cables expression correlated with a lack of Cables mRNA. Protein lysates were also generated from these same samples and evaluated by western blot. In a subset of these tissues, a loss of Cables expression was correlated with a loss of Cables mRNA.

HES and SK-UT2 tumors generated in nude mice were analyzed to verify whether in vitro observations mimicked tumor behavior observed in vivo.

Preparation of an Adenoviral Vector Construct Containing Cables

An adenoviral construct was generated which contains the Cables nucleic acid sequence. The Ad E1 region of adenovirus (serotype 5) was replaced with an MD expression cassette. Once incorporated into the MD expression cassette, Cables was placed under the control of the hCMV IE gene promoter. In addition, the construct also contained the human beta-globin IVS II sequence and a polyA signal sequence.

Ad-Cables virus was prepared as follows: virus was amplified from a single plaque by serial infection on 293A cells, and purified by two cycles of CsCl (cesium chloride) gradient centrifugation. The final products were titered using an optical absorbance method (Maizel et al. Virol. 36:115-125, 1968), and the plaque forming units/mL were determined. The purified viral preparation was stored in the following formulation: 50 mM Tris-HCl pH 7.4, 5 mM EDTA, 1.4 M CsCl, 50 mM NaCl, 0.5 MgCl2, and 25% glycerol. The viral preparation was found to be very stable at −20 C.

Diagnosis of a Cancer-Related Condition Using Cables

The present discovery that Cables controls cell growth and that the absence of Cables correlates with pre-cancerous and cancerous conditions facilitates novel assays for diagnosing subjects with a cancer-related condition, or a propensity toward developing a cancer-related condition. Those skilled in the art will appreciate that many different types of diagnostic assays are available for cancer detection. For example, proliferative disorders, such as cancers, may be correlated with particular mutations in the DNA of a patient. By comparing the sequence for a particular gene in both normal and tumor tissue from the same patient, one can determine if the mutation is of somatic or germline origin. This information may be used to screen a population as a whole for individuals that are at an increased risk of developing a particular type of proliferative disorder, or may be used to test individual patients, for example, those with family histories of cancerous conditions.

As described herein, Cables has been shown to function as a tumor suppressor for various cancer-related conditions, including prostate cancer, ovarian cancer, colorectal cancer, stomach cancer, lung cancer, esophageal cancer, head cancer, neck cancer, bladder cancer, squamous cell cancer, breast cancer, cervical cancer, and endometrial cancer. These results come from directly screening a number of head and neck carcinomas, squamous cell carcinomas, ovarian carcinomas, and endometrial carcinomas for expression of Cables and for mutations in the gene. Approximately 60% of the tumors examined were negative for Cables expression or showed altered or reduced patterns of expression by staining. Immunolocalization in normal tissue sections showed strong Cables staining in the nuclei of cells. In most instances, tumor cells showed a complete lack of staining. Cytoplasmic rather than nuclear staining was also seen in some areas of otherwise negative tumors.

In view of these results, diagnosis of a cancer-related condition in a patient using Cables can be performed by assaying for the expression or activity of a Cables gene (see below), or by determining the presence of a genetic lesion, such as mutations in the Cables gene or the complete absence of the genetic locus; in humans, this locus is positioned at chromosome 18q11-12. A genetic lesion can be determined by detecting the presence or absence of the Cables gene, gene expression, or polypeptide activity in a cell. This type of information may even be used to further characterize the cancer cell (e.g., to grade the stage to which the cancer has progressed) or to determine the prognosis of a patient who has been diagnosed with a cancer-related condition.

As noted above, a genetic lesion in the Cables gene may be associated with a cancer-related condition, for example, a proliferative disease. Specifically, this genetic lesion, resulting in the mutation of or loss of the Cables gene, can be identified from tissue samples from patients with cancer-related conditions, such as prostate cancer, ovarian cancer, colorectal cancer, stomach cancer, lung cancer, esophageal cancer, neck cancer, head cancer, bladder cancer, squamous cell cancer, breast cancer, cervical cancer, and endometrial cancer. Probes and primers based on the Cables gene sequence or based on known mutations in the Cables gene sequence can be used as markers to detect any mutation or loss of the Cables gene in samples from patients. Probes or primers may be based on the human Cables nucleic acid and amino acid sequences presented in FIGS. 9A-9B (SEQ ID NOS: 1 and 2), and genetic lesions may be identified by comparison with those sequences.

A genetic lesion in the Cables gene may be identified in a biological sample obtained from a patient using a variety of methods available to those skilled in the art. Generally, these techniques involve, for example, PCR amplification of nucleic acid from the patient sample, followed by identification of the genetic lesion by either altered hybridization, aberrant electrophoretic gel migration, restriction fragment length polymorphism (RFLP) analysis, binding or cleavage mediated by mismatch binding proteins, or direct nucleic acid sequencing. Any of these techniques may be used to facilitate detection of a genetic lesion in the Cables gene, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236 (1989)).

Alternatively, a genetic lesion in the Cables gene may be assayed by detecting changes in Cables expression, either at the RNA or protein levels. For example, expression of the Cables gene in a biological sample (e.g., a biopsy) may be monitored by standard northern blot analysis (to examine mRNA levels) or may be aided by PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294, 1991).

In yet another alternative, antibodies directed against a Cables protein (for example, those described herein) may be used to detect altered expression levels of the protein, including a lack of expression, or a change in its mobility on a gel, indicating a change in structure or size. In addition, antibodies may be used for detecting an alteration in the expression pattern or the sub-cellular localization of the protein (for example, the absence of Cables protein from the nuclear compartment). The antibody may be used in immunoassays to detect or monitor protein expression, e.g., Cables protein expression, in a biological sample. The antibody can be labeled, if desired, and used in standard immunoassays. A polyclonal or monoclonal antibody (produced as described below) may be used in any standard immunoassay format (e.g., ELISA, western blot, or RIA) to measure polypeptide levels. These levels may be compared to normal levels. Examples of immunoassays are described, e.g., in Ausubel et al. (supra).

Antibodies used in the present invention may include ones that recognize both the wild-type and mutant protein, as well as ones that are specific for either the wild-type or an altered form of the protein, for example, one encoded by a polymorphic or mutant Cables gene. Monoclonal antibodies may be prepared using the Cables protein described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, New York, N.Y., 1981; and Ausubel et al., supra). Once produced, monoclonal antibodies are also tested for specific Cables protein recognition by western blot or immunoprecipitation analysis (by the methods described in, for example, Ausubel et al., supra). Polyclonal antibodies that recognize wild type and mutant or polymorphic Cables protein can also be generated, for example, in rabbits, goats, or mice, and used in the present invention for diagnosis of a cancer-related condition.

Antibodies used in the methods of the invention may be produced using amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf (CABIOS 4:181, 1988). These fragments can be generated by standard techniques, e.g., by PCR, and cloned into an expression vector, for example pGEX (Ausubel et al., supra). GST fusion proteins can be made and expressed in E. coli and purified using a glutathione agarose affinity matrix as described in Ausubel et al. (supra).

Antibodies or oligonucleotide probes/primers can be utilized in methods that are known to one skilled in the art to examine biopsied tissue samples for the diagnosis of a cancer-related condition, e.g., to diagnose precancerous conditions, early stage cancers, and so forth. Immunohistochemical techniques may also be utilized for protein detection. For example, a tissue sample may be obtained from a patient, sectioned, and stained for the presence of Cables using an anti-Cables antibody and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982; and Ausubel et al., supra). This type of technique is particularly useful for detecting inappropriate cellular localization of the Cables protein (for example, for detecting the absence of Cables protein in the nucleus of sample cells).

Diagnosis of a cancer-related condition can also be accomplished by determining the biological activity of the Cables polypeptide. Cables biological activity includes, for example, interaction of Cables with other proteins, and this can be verified by numerous methods known to those skilled in the art, including, for example, co-immunoprecipitation assays. Alternatively, biological activity may be measured by assaying for protein phosphorylation, for example, examination of Cables biological activity in the context of Wee1-mediated tyrosine phosphorylation of cdk2. General guidance regarding these techniques can be found in standard laboratory manuals, such as Ausubel et al., supra, and Sambrook et al., Molecular Cloning: A Laboratory Manual, Cold Spring Harbor Laboratory, N.Y., (1989), and as described herein.

Methods for the diagnosis of a cancer-related condition according to the invention also include the detection of hypermethylation of CpG islands within the promoter of a Cables gene. Covalent modification of cellular substrates with methyl groups has been implicated in the pathology of cancer and other diseases (Gloria et al., Cancer 78:2300-2306, 1996). Cytosine hypermethylation of eukaryotic DNA prevents transcriptional activation (Turker and Bestor, Mutat. Res. 386:119-130, 1997). Hypermethylation at CpG islands of tumor suppressor genes is known to silence their expression in tumorigenesis. One skilled in the art can use methodologies described in, for example, Herman and Baylin, Methylation Specific PCR, in Current Protocols in Human Genetics, 1998, to identify the presence of hypermethylation of CpG islands within the promoter of a Cables gene.

Our results also indicate that Cables expression is hormonally regulated. This aspect of Cables expression can also be used to diagnose a cancer-related condition. For example, Cables expression is up-regulated by progesterone and down-regulated by estrogen. Therefore, a biopsy sample can be removed from a patient thought to have a cancer-related condition and examined for a response to progestin. If the tissue sample responds to progestin (i.e., exhibits an up-regulation of Cables expression or exhibits cell growth inhibition), a favorable diagnosis is warranted. If the sample is not responsive to progestin (i.e., no upregulation of Cables expression or cell growth inhibition), diagnosis of a cancer-related condition with a poorer prognosis can be made.

Prognosis for Treatment of a Cancer-Related Condition Using Cables

The present invention also features methods for determining the prognosis for treatment of a cancer-related condition in a subject. These methods utilize the techniques described above (i.e., determining the amount or location of Cables mRNA or polypeptide in a subject, detecting the presence of a mutation in or the absence of a Cables gene in a subject, or detecting the presence of hypermethylation of CpG islands within a promoter of the subject's Cables gene). Generally, a decrease in the amount of a Cables mRNA or polypeptide, as detected by the methods discussed above, indicates a negative prognosis for the treatment of a cancer-related condition. For example, the absence of functional Cables polypeptide or the lack of a sufficient amount of biologically active Cables polypeptide indicates that cell cycle regulation in a tumor cell of a subject (as determined, for instance, by Wee1-mediated tyrosine phosphorylation of cdk2) is defective. The level of biologically active Cables polypeptide can be determined by comparing the amount of biological activity of a Cables polypeptide in several subjects. Similarly, the determination of a loss or disruption of a Cables gene also indicates a negative prognosis, due to loss of Cables biological activity. Finally, a determination of hypermethylation of CpG islands in a Cables promoter of a cell of a subject also indicates a negative prognosis, since hypermethylation is known in the art to silence gene transcription, reducing or preventing expression of Cables protein necessary for cell cycle regulation.

The hormonal regulation of Cables by progesterone and estrogen can also be used to evaluate the prognosis for treatment of a cancer-related condition in a subject. As is described above, a biopsy sample can be taken from a patient thought to have a cancer-related condition and examined for a response to progestin. A positive response of the tissue sample to progestin (i.e., the cells of the tissue sample exhibit an up-regulation of Cables expression or exhibit cell growth inhibition), indicates a favorable prognosis for treatment of a cancer-related condition. A tissue sample that is non-responsive or poorly responsive to progestin (i.e., the cells of the sample exhibit little or no upregulation of Cables expression or cell growth inhibition) indicates a poor prognosis for treatment of the cancer-related condition.

Identification of a Candidate Compound for the Treatment, Stabilization, or Prevention of a Cancer-Related Condition

A candidate compound that is beneficial in the treatment, stabilization, or prevention of a cancer-related condition can also be identified by the methods of the present invention. A candidate compound can be identified by its ability to affect the biological activity of a Cables polypeptide or the expression of a Cables gene. Compounds that are identified by the methods of the present invention that increase the biological activity or expression levels of a Cables polypeptide or that compensate for the loss of Cables polypeptide activity or gene expression, for example, due to loss of the Cables gene due to a genetic lesion, represent candidate compounds or lead compounds for the treatment, stabilization, or prevention of a cancer-related condition. A candidate compound identified by the present invention can increase the biological activity of a Cables polypeptide, for example, by increasing the Wee1-mediated tyrosine phosphorylation of cdk2. A candidate compound identified by the methods of the present invention can also, for example, increase the expression of a Cables gene, either by increasing transcription of the Cables gene or translation of the Cables mRNA, or it can increase nuclear localization of the Cables polypeptide.

Expression of a reporter construct that is operably linked to a Cables promoter can be used to identify such candidate compounds. A reporter construct may encode a reporter enzyme that has a detectable read-out, such as beta-lactamase, beta-galactosidase, or luciferase. Reporter enzymes can be detected using methods known in the art, such as the use of chromogenic or fluorogenic substrates for reporter enzymes as such substrates are known in the art. Such substrates are desirably membrane permeant. Chromogenic or fluorogenic readouts can be detected using, for example, optical methods such as absorbance or fluorescence. A reporter construct can be incorporated into a plasmid or viral vector, for example, a retrovirus or adeno-associated virus. A reporter construct can also be extra-chromosomal or be integrated into the genome of a host cell. The expression of the reporter construct can be under the control of exogenous expression control sequences or expression control sequences within the genome of the host cell. Under the latter configuration, the reporter construct is desirably integrated into the genome of the host cell.

A candidate compound identified by the methods of the present invention can be from natural as well as synthetic sources. Those skilled in the field or drug discovery and development will understand that the precise source of test extracts or compounds is not critical to the methods of the invention. Examples of such extracts or compounds include, but are not limited to, plant-, fungal-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Screening methods according to the invention may be carried out in any cell, for example, a cell (such as a mammalian cell) into which a heterologous Cables gene or a Cables reporter construct has been introduced. Alternatively, these screens may be carried out in cells in which the Cables gene is defective, has reduced activity or expression or inappropriate cellular localization, or is non-functional. In these cells, compounds that increase or compensate for Cables activity can be identified, as compounds that either increase the low level of Cables expression or activity, increase nuclear Cables localization, or allow the cells to return to near or completely normal phenotypes. Phenotypes that may be assayed include, without limitation, cdk2 phosphorylation (as described herein), Cables expression, activity, or localization (as described herein), or cell proliferation. Desirable candidate compounds are identified as those which increase phosphorylation, increase Cables expression, activity, or nuclear localization, or decrease cell proliferation, as compared to those phenotypes in cells carrying the Cables mutation or other genetic lesion.

Use of an Animal Model to Identify a Cables-Related Candidate Compound

The present invention also provides methods for using a transgenic, knockout, or mutant animal that develops a cancer-related condition and can accurately recapitulate many of the features of the cancer-related condition associated with loss or mutation of the Cables gene. Without limitation, a particularly desired transgenic, knockout, or mutant animal is one in which the tumorigenic phenotype is fully penetrant, the rate of progression of the neoplasm is rapid, and/or the lifespan of the transgenic or knockout animal is not shortened by a knockout- or transgene-related pathology in an organ. Of course, it will be appreciated that these traits are not required. Desirably, the Cables gene is used to produce the transgenic animal or the Cables gene is the target of the knockout.

A transgenic animal expressing a mutant Cables gene can be used to identify a candidate compound that is useful for the treatment, stabilization, or prevention of a cancer-related condition. Transgenic animals expressing a conditional mutant Cables gene (e.g., using a tetracycline regulatable system) can also be generated and is well known to those skilled in the art and is described in, for example, WO 94/29442, WO 96/40892, WO 96/01313, and Yamamoto et al. (Cell 101:57-66, 2000). In addition, the knockout animal may be a conditional knockout using, for example, the FLP/FRT system described in, for example, U.S. Pat. No. 5,527,695, and in Lyznik et al. (Nucleic Acid Research 24:3784-3789, 1996) or the Cre-lox recombination system described, for example, in Kilby et al. (Trends in Genetics 9:413-421, 1993).

Transgenic animals may be made using standard techniques, such as those described in Sambrook et al., Molecular Cloning: A Laboratory Manual, Cold Spring Harbor Laboratory, N.Y., 1989). For example, a Cables transgene may be constructed using endogenous control sequences or using constitutive, tissue-specific, or inducible regulatory sequences. Any tissue specific promoter may direct the expression of any Cables protein used in the invention, such as prostate-, ovarian-, colon-, stomach-, lung-, esophageal-, head-, neck-, bladder-, squamous cell-, breast-, cervical-, or endometrial-specific promoters. For example, a mutated gene may be used to replace the wild type Cables gene.

A transgenic or knockout animal, as disclosed herein, may be used as a research tool to determine genetic and physiological features of a cancer-related condition, or for identifying compounds that can affect tumors. A knockout animals also include an animal in which the normal gene has been inactivated or removed and replaced with a polymorphic allele of this gene. The animal can serve as a model system for the risk of developing, treating, stabilizing, or preventing a cancer-related condition that is associated with a Cables gene polymorphism or mutation.

In general, a transgenic or knockout animal can be used to identify a candidate compound useful for treating, stabilizing, or preventing a cancer-related condition. The candidate compound is identified by contacting the transgenic or knockout animal with the candidate compound and comparing the presence, absence, or level of expression of a Cables gene or a Cables-related gene, either at the RNA level or at the protein level, in tissue derived from a transgenic or knockout animal, as is described above, as well as in tissue derived from a matching non-transgenic or knockout animal. Standard techniques for detecting RNA expression, e.g., by northern blotting, or protein expression, e.g., by western blotting, are well known in the art. Alternatively, the effect of a candidate compound may be assayed by phosphorylation assays, as described herein. The response to or progression of disease in a transgenic or knockout animal, as compared with non-transgenic or knockout animals can be used to identify compounds that may be effective therapeutics against a cancer-related condition, such as prostate tumors, ovarian tumors, colorectal tumors, stomach tumors, lung tumors, esophageal tumors, head tumors, neck tumors, bladder tumors, squamous cell tumors, breast tumors, cervical tumors, or endometrial tumors. Transgenic and knockout animals can also be used to predict whether compounds identified as therapeutics will affect disease progression.

As an alternative to a transgenic or knockout animal, a mutant version of any mammal that naturally expresses Cables may also be utilized for any of the above applications. Such animals may harbor a mutation that decreases or eliminates Cables expression or activity, preferably by at least 10%, more preferably, by at least 30%, and most preferably, by at least 50%, 70%, 85%, 90%, or even 95% or 100% of the naturally-occuring level of expression or activity. Conversely, animals may be utilized that increase Cables expression or activity, preferably by at least 2-fold, preferably, 5-fold, and more preferably, buy 10-fold or more. These animals may be generated by any standard method of mutagenesis.

Any transgenic animal, or cells derived from these animals, may be constructed and used for compound screening. Preferable animal models include, without limitation, monkeys, pigs, goats, sheep, cats, dogs, rodents (for example, mice, rats, and rabbits), flies, and nematodes.

Administration of Cables or a Candidate Compound for the Treatment, Stabilization, or Prevention of a Cancer-Related Condition

The present invention further includes methods for treating, stabilizing, or preventing a cancer-related condition by administering a Cables polypeptide, or a biologically-active fragment thereof, or a compound that enhances Cables expression, activity, or nuclear localization. The administration of a biologically active Cables polypeptide, or fragment thereof, that, regardless of its method of manufacture, retains full biological activity, can be utilized to restore Cables biological activity in a patient lacking endogenous Cables due to a loss or reduction of its expression or biological activity (for example, by mutation or loss of a Cables gene). Alternatively, a compound that compensates for or enhances Cables expression or activity can be similarly used.

Peptide agents of the invention, such as a Cables polypeptide or biologically-active fragment thereof, or a candidate compound can be administered to a subject, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (18th edition), ed. A. Gennaro, 1990, Mack Publishing Company, Easton, Pa.

Pharmaceutical formulations of a therapeutically effective amount of a peptide agent or candidate compound of the invention, or pharmaceutically acceptable salt-thereof, can be administered orally, parenterally (e.g., intramuscularly, intraperitoneally, intravenously, or intradermally; by subcutaneous injection; by inhalation; or through the use of optical drops or an implant), nasally, vaginally, rectally, sublingually, or topically, in admixture with a pharmaceutically acceptable carrier adapted for the route of administration.

Methods well known in the art for making formulations are found, for example, in Remington's Pharmaceutical Sciences (18th edition), ed. A. Gennaro, 1990, Mack Publishing Company, Easton, Pa. Compositions intended for oral use may be prepared in solid or liquid forms according to any method known to the art for the manufacture of pharmaceutical compositions. The composition may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

Compositions for rectal or vaginal administration are desirably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the polypeptide being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

The polypeptide or candidate compound of the invention can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be desired over a prolonged release composition. Alternatively, for preventative or long-term treatments, a sustained released composition will generally be desired.

The polypeptide or candidate compound of the present invention can be prepared in any suitable manner. The polypeptide or candidate compound can be isolated from a naturally occurring source, recombinantly or synthetically produced, or produced by a combination of these methods. The synthesis of short peptides is well known in the art. See, e.g., Stewart et al., Solid Phase Peptide Synthesis (Pierce Chemical Co., 2d ed., 1984).

Adminstration of Progesterone in Combination with Cables for the Treatment, Stabilization, or Prevention of a Cancer-Related Condition

Progesterone can be administered in combination with a Cables polypeptide or nucleic acid molecule, or in combination with a compound that increases Cables polypeptide expression or biological activity for the treatment or prevention of a cancer-related condition in a subject. Progesterone is always administered as a progestin (also called a progestagen), which is a synthetic progesterone-like compound. Medroxyprogesterone (PROVERA) is used most commonly today, but there are others.

Progestin can be administered by injection, orally, transdermally, sublingually, or by cream or suppository. The dosage of progestin depends on several factors, including: the administration method, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Generally, when systemically administered to a human (e.g., by oral, intramuscular, subcutaneous, topical, inhalation, transdermal, sublingual, rectal, or vaginal administration), the dosage of the progestein is normally about 0.001 mg to 200 mg per day, desirably about 1 mg to 100 mg per day, and more desirably about 5 mg to 25 mg per day. Dosages up to 200 mg per day may be necessary. For intravenous administration of the progestin, the dosage is normally about 1 mg to 200 mg per day, desirably about 10 mg to 150 mg per day, and more desirably about 25 mg to 50 mg per day. Systemic dosing will result in steady-state plasma concentrations of progestin of desirably 0.1 μM to 7.0 μM, more desirably, 0.5 μM to 5.0 μM, and most desirably, 1.0 μM to2.0 μM.

The dosage, frequency, and mode of administration of each component of the combination can be controlled independently. For example, progestin may be administered orally one time per day, while the second component may be administered topically three times per day. Combination therapy may be given in on-and-off cycles that include rest periods so that the patient's body has a chance to recover from any as yet unforeseen side-effects. The compounds may also be formulated together such that one administration delivers both compounds.

Administration of the progestin, in combination with a Cables polypeptide or nucleic acid molecule, or in combination with a compound that increases Cables polypeptide expression or biological activity, can be one to four times daily for one day to one year, and may even be for the life of the patient. Chronic, long-term administration will be indicated in many cases.

Cables Gene Therapy

In yet another embodiment of the invention, the Cables gene may also be administered to a subject using gene therapy techniques. See, generally, Morgan et al., Ann. Rev. Biochem. 62:191-217, 1993; Culver et al., Trends Genet. 10:174-178, 1994; and U.S. Pat. No. 5,399,346 (French et al.). The general principle is to introduce the Cables gene, for example, into a cancer cell in a patient, such that the Cables gene is expressed and produces a Cables polypeptide, or a biologically-active fragment thereof, that can supplement the activity of the endogenous, defective, or absent Cables polypeptide.

A desired mode of gene therapy is to provide the Cables polynucleotide in such a way that it will replicate inside the cell, thereby enhancing and prolonging the interference effect. Thus, the Cables polynucleotide can be operably linked to a suitable promoter, such as the natural promoter of the corresponding gene, a heterologous promoter that is intrinsically active in cancer cells, or a heterologous promoter that can be induced by a suitable agent.

In another aspect of gene therapy according to the invention, a polynucleotide is introduced into a cancer cell such that the polynucleotide interferes with the expression of a Cables-related gene, for example, a gene involved in cell cycle regulation (e.g., cdk2). The administered polynucleotide blocks expression of the Cables-related gene by forming a complex with the Cables-related gene directly, or by complexing with the RNA transcribed from the Cables-related gene. Desirably, the construct is designed so that the polynucleotide sequence is complementary to the sequence of the Cables-related gene. Thus, once integrated into the cellular genome, the transcript of the administered polynucleotide will be complementary to the transcript of the Cables-related gene, and therefore, the polynucleotide will be capable of hybridizing with the Cables-related gene transcript. This approach is known as anti-sense therapy or RNAi. See, for example, Culver et al., supra; and Roth, Ann. Surg. Oncol.1:79-86, 1994.

Exemplary disease targets include, but are not limited to, prostate cancer, ovarian cancer, colorectal cancer, stomach cancer, lung cancer, esophageal cancer, head cancer, neck cancer, bladder cancer, squamous cell cancer, breast cancer, cervical cancer, and endometrial cancer.

For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, supra. Methods commonly known in the art of recombinant DNA technology that can be used are described in Ausubel et al. supra; and Kriegler, 1990, Gene Transfer and Expression, A Laboratory Manual, Stockton Press, NY.

It is envisioned that a patient that has been diagnosed with, or that has a propensity for developing, a cancer-related condition can be administered a Cables gene, using a suitable method known in the art and as described herein, such that the Cables gene is incorporated into one or more cells of the patient and is expressible by the cell(s) and/or progeny of the cell(s). The method can encompass in vivo administration of the Cables gene in a suitable composition, or the method can involve ex vivo therapy in which one or more cells of the patient are removed, transformed with the Cables gene, optionally expanded, and readministered to the patient. Expression of the Cables gene in the transformed cells will reactivate Cables activity in the patient, thereby promoting regulation of the cell cycle, as is discussed above, and therefore, inhibition of the cancer-related condition, thus leading to improvement of the diseased condition afflicting the patient.

Transformation of a target cell with a Cables nucleic acid molecule is facilitated by suitable techniques known in the art, such as providing the Cables nucleic acid molecule in the form of a suitable vector, or encapsulation of the Cables nucleic acid molecule in a liposome. The nucleic acid molecule may be provided to the cancer site by an antigen-specific homing mechanism, or by direct injection. In one approach, the nucleic acid molecule is operably linked to a promoter and is contained in an expression vector. In another approach, the nucleic acid molecule is contained in a recombinant viral vector, for example an adenoviral vector (see e.g., Kozarsky and Wilson, Current Opinion in Genetics and Development 3:499-503, 1993; Rosenfeld et al., Science 252:431-434, 1991; Rosenfeld et al., Cell 68:143-155, 1992; and Mastrangeli et al., J. Clin. Invest. 91:225-234, 1993), an adeno-associated viral vector (AAV; see, for example, Walsh et al., Proc. Soc. Exp. Biol. Med. 204:289-300, 1993), a lentiviral vector, a herpes viral vector, a retroviral vector (see, e.g., Miller et al., 1993, Meth. Enzymol. 217:581-599; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), a pox virus vector, or a baculoviral vector.

Non-viral vectors can also be used for gene therapy. For example, naked DNA can be delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, or pressure-mediated gene delivery. Numerous techniques are known in the art for the introduction of foreign genes into cells (see, e.g., Loeffler and Behr, Meth. Enzymol. 217:599-618, 1993; Cohen et al., Meth. Enzymol. 217:618-644, 1993; Cline, Pharmac. Ther. 29:69-92, 1985), and may be used in accordance with the present invention, provided that the necessary developmental and physiological functions of the recipient cells are not disrupted. Usually, the method of transfer includes the transfer of a selectable marker to the cells. The cells are then placed under selection to isolate those cells that have taken up and are expressing the transferred gene. Those transformed cells are then delivered to a patient. The technique should provide for the stable transfer of the gene to the cell, so that the gene is expressible by the cell and preferably heritable and expressible by progeny of the cell.

Preferably, a desired gene is introduced intracellularly and incorporated within the host precursor cell DNA for expression, by homologous recombination (see, e.g., Koller and Smithies, Proc. Natl. Acad. Sci. USA 86:8932-8935, 1989; Zijlstra et al., Nature 342:435-438, 1989).

The vector containing the Cables gene, or a fragment thereof, can be administered as is described above for the administration of a peptide agent or candidate compound of the invention, for example, to an artery at the site of a tumor or other cancerous cell.

Various reports have been presented regarding the efficacy of gene therapy for the treatment of monogeneic diseases, early stage tumors, and cardiovascular disease. (See, e.g., Blaese et al., Science 270:475-480, 1995; Wingo et al., Cancer 82:1197-1207, 1998; Dzao, Keystone Symposium Molecular and Cellular Biology of Gene Therapy, Keystone, Co. Jan. 19-25, 1998; and Isner, Keystone Symposium Molecular and Cellular Biology of Gene Therapy, Keystone, Co. Jan. 19-25, 1998.)

In a preferred embodiment, patients diagnosed with prostate cancer, ovarian cancer, colorectal cancer (e.g., colorectal adenocarcinoma), stomach cancer, lung cancer, esophageal cancer, head cancer, neck cancer, bladder cancer (e.g., bladder transitional cell carcinoma), squamous cell cancer, breast cancer, cervical cancer, or endometrial cancer can be treated using in vivo methods consisting of the administration of a recombinant retrovirus containing a Cables cDNA under the control of a promoter (e.g., a prostate-, ovary-, colon-, stomach-, lung-, esophageal-, head-, neck-, bladder-, squamous cell-, breast-, cervical-, or endometrial-specific promoter) for expression in tumor cells. In vivo therapy involves transfection of a Cables nucleic acid molecule directly into the cells of a patient without the need for prior removal of those cells from the patient.

In vivo delivery is desirably accomplished by (1) infusing a recombinant retrovirus vector construct into a blood vessel that perfuses the tumor or (2) injecting a recombinant retrovirus vector construct directly into the tumor. In an especially desired in vivo embodiment, a catheter is inserted into a blood vessel in the neck of an organism and the tip of the indwelling catheter is advanced with fluoroscopic guidance to a position in an artery that perfuses a portion of the tumor. It is desired that the tip of an indwelling catheter be placed in proximity to an area of the tumor so that the cells can be directly targeted and transfected. The retroviral construct can also be directly targeted to cancer cells using cancer cell-specific surface antigens, although this is not required. The recombinant retrovirus is administered to patients desirably by means of intravenous administration in any suitable pharmacological composition, either as a bolus or as an infusion over a period of time. Injection of the recombinant retrovirus directly into the tumor, or into a blood vessel that perfuses the tumor will promote incorporation of the Cables cDNA into tumor cells, thereby inhibiting cell growth of the tumor and preventing further tumor formation.

After delivery of a recombinant retrovirus vector construct to the cells of the tumor, the cells are maintained under physiological conditions to allow sufficient time for the retrovirus vector construct to infect the cancer cells and for cellular expression of the Cables polypeptide contained in that construct. A time period sufficient for expression of a Cables polypeptide in a cancer cell varies as is well known in the art depending on the type of retrovirus vector used and the method of delivery. It should also be pointed out that because that the retrovirus vector employed may be replication defective, it may not be capable of replicating in the cells that are ultimately infected.

A retrovirus vector construct is typically delivered in the form of a pharmacological composition that comprises a physiologically acceptable carrier and the retrovirus vector construct. An effective amount of a retrovirus vector construct is delivered, and consists of 1 pfu/cell, 5 pfu/cell, 10 pfu/cell, or 20 pfu/cell, or any other amount that is effective for promoting expression of a Cables polypeptide in the target cancer cells. Means for determining an effective amount of a retrovirus vector construct are well known in the art.

As is also well known in the art, a specific dose level for any particular subject depends upon a variety of factors including the infectivity of the retrovirus vector, the age, body weight, general health, sex, diet, time of administration, route of administration, rate of excretion, and the severity of the condition of the patient.

In one exemplary approach, an adenoviral construct containing Cables (Ad-Cables), which promotes overexpression of Cables in cell lines transformed with the construct, can be used to inhibit cell proliferation and promote cell death. To demonstrate the efficiency of this approach, HES cells were infected with the Ad-Cables construct and, following a 48 hour incubation, dose response and time course experiments were performed. HES cells were infected with an MOI of 0.5 to 100. An MOI of <20 resulted in the lack of any observable cytopathic effect.

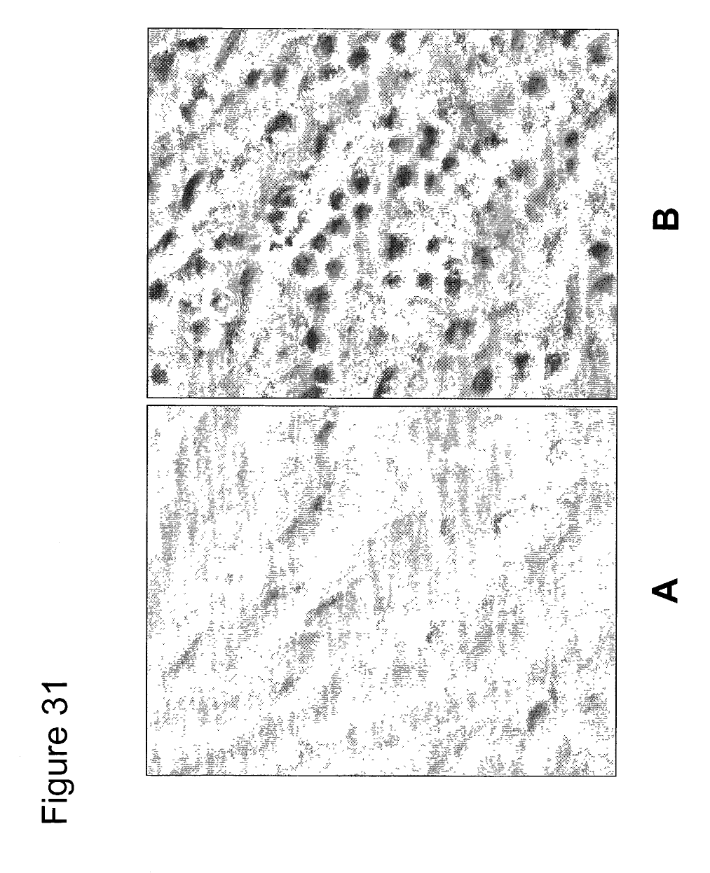

The expression level of Cables in HES cells transformed with Ad-Cables was compared to the expression level of HES cells transformed with a control adenovirus containing a GFP tag. HES cells infected with Ad-Cables at a multiplicity of infection of 1 (MOI=1) demonstrated significant inhibition of cell proliferation and evidence of cell death (FIG. 31A) as compared to control HES cells infected with the Ad-control vector or the parent HES cell line (FIG. 31B). There was no difference in the cellular proliferation rate in the HES cells containing the Ad-control or the parent cell line. These results support the use of retroviral vector-mediated transformation of cells with Cables for use in gene therapy techniques for the treatment of a cancer-related condition in a patient. Furthermore, Cables expression via adenoviral therapy can be combined with progestin treatment to increase the therapeutic effect of Cables expression.

Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth. All publications and patent applications mentioned in this specification are herein incorporated by reference to the same extent as if each independent publication or patent application was specifically and individually indicated to be incorporated by reference.

Other embodiments are within the claims.

What is claimed is:

1. A method of diagnosing a cancer-related condition or a propensity thereto in a subject, said method comprising measuring Cables expression or Cables activity in a sample from said subject, wherein a decrease in said Cables expression or Cables activity in said sample, relative to the amount of Cables expression or Cables activity in a control sample, indicates that said subject has said cancer-related condition or a propensity thereto.

2. The method of claim 1, wherein said sample further comprises a cdk2 polypeptide or a Wee1 polypeptide.

3. The method of claim 2, wherein said sample further comprises a cdk2 polypeptide and a Wee1 polypeptide.

4. The method of claim 3, wherein said Cables activity is determined by measuring Wee1-mediated tyrosine phosphorylation of cdk2.

5. The method of claim 1, wherein said Cables expression is determined by measuring the amount of Cables mRNA or polypeptide in said sample from said subject.

6. The method of claim 1, wherein said Cables expression or Cables activity in said sample is compared to Cables expression or Cables activity in control samples from subjects having said cancer-related condition and subjects not having said cancer-related condition.

7. A method of diagnosing a cancer-related condition or a propensity thereto in a subject, said method comprising detecting the absence of a Cables gene or a mutation in a Cables gene in said subject, wherein the absence of said Cables gene or a mutation in said Cables gene indicates a cancer-related condition or a propensity thereto in said subject.

8. The method of claim 7, wherein said Cables gene is located on chromosome 18, region q11.2-q12.1.

9. The method of claim 7, wherein said mutation in the Cables gene results in expression of a truncated Cables polypeptide.

10. A method of diagnosing a cancer-related condition or a propensity thereto in a subject, said method comprising detecting the presence of hypermethylation of CpG islands in a Cables promoter of said subject, wherein the presence of said hypermethylation indicates a cancer-related condition or a propensity thereto in said subject.

11. The method of any of claims 1, 7, or 10, wherein said subject is a mammal.

12. The method of claim 11, wherein said mammal is a human.

13. The method of any of claims 1, 7, or 10, wherein said cancer-related condition is prostate cancer, ovarian cancer, colorectal cancer, stomach cancer, lung cancer, esophageal cancer, head cancer, neck cancer, bladder cancer, squamous cell cancer, or endometrial cancer.

14. The method of any of claims 1, 7, or 10, wherein said cancer-related condition is endometrial cancer.

15. A method of identifying a subject at increased risk of developing a cancer-related condition, said method comprising the step of identifying an alteration in a Cables nucleic acid molecule of said subject, wherein the presence of said alteration indicates that said subject has an increased risk of developing a cancer-related condition.

16. The method of claim 15, wherein said subject is a mammal.

17. The method of claim 16, wherein said mammal is a human.

18. The method of claim 15, wherein said cancer-related condition is prostate cancer, ovarian cancer, colorectal cancer, stomach cancer, lung cancer, esophageal cancer, head cancer, neck cancer, bladder cancer, squamous cell cancer, or endometrial cancer.

19. The method of claim 15, wherein said cancer-related condition is endometrial cancer.

20. A method for determining the prognosis for treatment of a cancer-related condition in a subject, said method comprising measuring the level of Cables expression or Cables activity in a sample from said subject, wherein an increase or decrease in said Cables expression or Cables activity in said sample, relative to the amount of Cables expression or Cables activity in a control sample, determines the prognosis for treatment of a cancer-related condition in said subject.

21. The method of claim 20, wherein the level of said Cables expression or Cables activity in said sample is compared to the level of Cables expression or Cables activity in control samples from subjects having said cancer-related condition and subjects not having said cancer-related condition.

22. The method of claim 20, wherein a decrease in said level of Cables expression or Cables activity indicates a negative prognosis for the treatment of said cancer-related condition.

23. A method for determining the prognosis for treatment of a cancer-related condition in a subject, said method comprising detecting the absence of a Cables gene or a mutation in a Cables gene in said subject, wherein the absence of said Cables gene or a mutation in said Cables gene indicates a negative prognosis for the treatment of said cancer-related condition in said subject.

24. The method of claim 20, wherein said Cables gene is located on chromosome 18, region q11.2-q12.1.

25. The method of claim 20, wherein said mutation in the Cables gene results in expression of a truncated Cables polypeptide.

26. A method for determining the prognosis for treatment of a cancer-related condition in a subject, said method comprising detecting the presence of hypermethylation of CpG islands in a Cables promoter of said subject, wherein the presence of said hypermethylation indicates a negative prognosis for said cancer-related condition.

27. The method of any of claims 20, 23, or 26, wherein said cancer-related condition is prostate cancer, ovarian cancer, colorectal cancer, stomach cancer, lung cancer, esophageal cancer, head cancer, neck cancer, bladder cancer, squamous cell cancer, or endometrial cancer.

28. The method of any of claims 20, 23, or 26, wherein said cancer-related condition is endometrial cancer.

29. The method of any of claims 20, 23, or 26, wherein said subject is a human.

30. A method for identifying a candidate compound for treating, stabilizing, or preventing a cancer-related condition, said method comprising the steps of:
(a) contacting a cell or in vitro sample comprising a Cables nucleic acid molecule or polypeptide with a candidate compound; and
(b) measuring a biological parameter of said Cables nucleic acid molecule or polypeptide, wherein an increase in said biological parameter, relative to the biological parameter of a Cables nucleic acid molecule or polypeptide in a control cell or in vitro sample not contacted with said compound, indicates a compound useful for treating, stabilizing, or preventing said cancer-related condition.

31. The method of claim 30, wherein said cell or in vitro sample comprises a Cables nucleic acid molecule, wherein measuring said biological parameter comprises measuring the level of transcription or translation of said Cables nucleic acid molecule.

32. The method of claim 30, wherein said cell or in vitro sample comprises a Cables polypeptide, wherein measuring said biological parameter comprises measuring the level of biological activity of said Cables polypeptide.

33. The method of claim 30, wherein said cell or in vitro sample further comprises a cdk2 polypeptide, a Wee1 polypeptide, or both.

34. The method of claim 33, wherein step (b) is carried out by measuring Wee1-mediated tyrosine phosphorylation of cdk2, wherein an increase in said phosphorylation, relative to the Wee1-mediated tyrosine phosphorylation of cdk2 in a control cell or in vitro sample not contacted with said compound, indicates a candidate compound for treating, stabilizing, or preventing said cancer-related condition.

35. The method of claim 34, wherein said tyrosine phosphorylation of cdk2 occurs at the tyrosine at position 15.

36. The method of claim 30, wherein step (b) further comprises measuring the amount of Cables mRNA or polypeptide in said cell or in vitro sample, wherein an increase in the amount of said Cables mRNA or polypeptide, relative to the amount of Cables mRNA or polypeptide in a control cell or in vitro sample not contacted with said compound, indicates a candidate compound for treating, stabilizing, or preventing said cancer-related condition.

37. The method of claim 30, wherein said cell is a transgenic cell expressing a heterologous Cables polypeptide.

38. The method of claim 37, wherein said transgenic cell is in a transgenic animal.

39. A method for identifying a candidate compound for treating, stabilizing, or preventing a cancer-related condition, said method comprising the steps of:
(a) contacting a cell or in vitro sample that expresses a Cables gene or Cables reporter construct with a candidate compound; and
(b) measuring the level of expression of said Cables gene or Cables reporter construct, wherein an increase in expression of said Cables gene or Cables reporter construct, relative to the expression of a Cables gene or Cables reporter construct in a control cell or in vitro sample not contacted with said compound, indicates a candidate compound for treating, stabilizing, or preventing said cancer-related condition.

40. The method of claim 39, wherein expression of said Cables gene or Cables reporter construct is measured by assaying the level of mRNA transcribed from said Cables gene or said Cables reporter construct.

41. The method of claim 39, wherein expression of said Cables gene or Cables reporter construct is measured by assaying the level of polypeptide translated from said Cables gene or said Cables reporter construct.

42. The method of claim 30 or 39, wherein said cancer-related condition is prostate cancer, ovarian cancer, colorectal cancer, stomach cancer, lung cancer, esophageal cancer, head cancer, neck cancer, bladder cancer, squamous cell cancer, or endometrial cancer.

43. The method of claim 30 or 39, wherein said cancer-related condition is endometrial cancer.

44. The method of claim 30 or 39, wherein said Cables gene or Cables polypeptide is or is derived from a human Cables gene or human Cables polypeptide.

45. A method of treating, stabilizing, or preventing a cancer-related condition in a subject, said method comprising administering a Cables polypeptide, or a fragment thereof, to said subject in an amount sufficient to treat, stabilize, or prevent said cancer-related condition.

46. A method of treating, stabilizing, or preventing a cancer-related condition in a subject, said method comprising administering a compound that increases Cables polypeptide expression or activity to said subject in an amount sufficient to treat, stabilize, or prevent said cancer-related condition.

47. A method of treating, stabilizing, or preventing a cancer-related condition in a subject, said method comprising administering a Cables polypeptide-expressing nucleic acid molecule to said subject in an amount sufficient to treat, stabilize, or prevent said cancer-related condition.

48. The method of claim 47, wherein said Cables polypeptide-expressing nucleic acid molecule comprises the sequence of human Cables as set forth in SEQ ID NO:1.

49. The method of claim 47, wherein said Cables polypeptide-expressing nucleic acid molecule is administered in an adenoviral vector construct.

50. The method of any one of claims 45, 46, or 47, wherein said cancer-related condition is prostate cancer, ovarian cancer, colorectal cancer, stomach cancer, lung cancer, esophageal cancer, head cancer, neck cancer, bladder cancer, squamous cell cancer, or endometrial cancer.

51. The method of any one of claims 45, 46, or 47, wherein said cancer-related condition is endometrial cancer.

52. The method of any one of claims 45, 46, or 47, wherein said subject is a human.

53. A substantially pure and isolated nucleic acid molecule comprising the nucleic acid sequence of SEQ ID NO: 1.

54. A substantially pure polypeptide comprising the amino acid sequence of SEQ ID NO: 2.
